SEQUENCE LISTING (120 sequences)

SEQ ID NO: 1 - protein, 5 aa, synthetic construct
DYGMS

SEQ ID NO: 2 - protein, 17 aa, synthetic construct
GIDWNGDSTGYADSVKG

SEQ ID NO: 3 - protein, 18 aa, synthetic construct
DQGIAVAGPYNYYYGMDV

SEQ ID NO: 4 - protein, 5 aa, synthetic construct
DYGMT

SEQ ID NO: 5 - protein, 17 aa, synthetic construct
GINWNGGSTGYADSVKG

SEQ ID NO: 6 - protein, 19 aa, synthetic construct
DQGIAVVGTHYYYYYGMDV

SEQ ID NO: 7 - protein, 5 aa, synthetic construct
YNYWS

SEQ ID NO: 8 - protein, 16 aa, synthetic construct
YIYHSGTTKYNPSLKS

SEQ ID NO: 9 - protein, 9 aa, synthetic construct
HEDYWFFDL

SEQ ID NO: 10 - protein, 5 aa, synthetic construct
DSGMS

SEQ ID NO: 11 - protein, 17 aa, synthetic construct
GINWNGGSTGYADSVKG

SEQ ID NO: 12 - protein, 19 aa, synthetic construct
DPGIAVAGPYYYYYYGMDV

SEQ ID NO: 13 - protein, 5 aa, synthetic construct
NYYWS

SEQ ID NO: 14 - protein, 16 aa, synthetic construct
YIYYSGSTNYNPSLKS

SEQ ID NO: 15 - protein, 9 aa, synthetic construct
HEDYWYFDL

SEQ ID NO: 16 - protein, 5 aa, synthetic construct
DYAMH

SEQ ID NO: 17 - protein, 17 aa, synthetic construct
LISGDDGSTYYADSMKG

SEQ ID NO: 18 - protein, 16 aa, synthetic construct
ASYGSGSYLYNYGMDV

SEQ ID NO: 19 - protein, 5 aa, synthetic construct
NAWMN

SEQ ID NO: 20 - protein, 19 aa, synthetic construct
RIKSKTDGGTKDYAAPVKG

SEQ ID NO: 21 - protein, 17 aa, synthetic construct
DPRIAVTGTDYYYGMDV

SEQ ID NO: 22 - protein, 5 aa, synthetic construct
SYAMH

SEQ ID NO: 23 - protein, 17 aa, synthetic construct
VISYDGSNKYYADSVKG

SEQ ID NO: 24 - protein, 21 aa, synthetic construct
APYYGSGSYYNSYYYYHGMDV

SEQ ID NO: 25 - protein, 5 aa, synthetic construct
NAWMN

SEQ ID NO: 26 - protein, 19 aa, synthetic construct
RIKSKTDGGTTDYAAPVKG

SEQ ID NO: 27 - protein, 17 aa, synthetic construct
DPRIAVTGTDYYYGMDV

SEQ ID NO: 28 - protein, 7 aa, synthetic construct
GFMFDDY

SEQ ID NO: 29 - protein, 6 aa, synthetic construct
DWNGDS

SEQ ID NO: 30 - protein, 18 aa, synthetic construct
DQGIAVAGPYNYYYGMDV

SEQ ID NO: 31 - protein, 7 aa, synthetic construct
GFTFDDY

SEQ ID NO: 32 - protein, 6 aa, synthetic construct
NWNGGS

SEQ ID NO: 33 - protein, 19 aa, synthetic construct
DQGIAVVGTHYYYYYGMDV

SEQ ID NO: 34 - protein, 7 aa, synthetic construct
GGSISYN

SEQ ID NO: 35 - protein, 5 aa, synthetic construct
YHSGT

SEQ ID NO: 36 - protein, 9 aa, synthetic construct
HEDYWFFDL

SEQ ID NO: 37 - protein, 7 aa, synthetic construct
GFTFDDS

SEQ ID NO: 38 - protein, 6 aa, synthetic construct
NWNGGS

SEQ ID NO: 39 - protein, 19 aa, synthetic construct
DPGIAVAGPYYYYYYGMDV

SEQ ID NO: 40 - protein, 7 aa, synthetic construct
GGSISNY

SEQ ID NO: 41 - protein, 5 aa, synthetic construct
YYSGS

SEQ ID NO: 42 - protein, 9 aa, synthetic construct
HEDYWYFDL

SEQ ID NO: 43 - protein, 7 aa, synthetic construct
GFTFDDY

SEQ ID NO: 44 - protein, 6 aa, synthetic construct
SGDDGS

SEQ ID NO: 45 - protein, 16 aa, synthetic construct
ASYGSGSYLYNYGMDV

SEQ ID NO: 46 - protein, 7 aa, synthetic construct
GFTFSNA

SEQ ID NO: 47 - protein, 8 aa, synthetic construct
KSKTDGGT

SEQ ID NO: 48 - protein, 17 aa, synthetic construct
DPRIAVTGTDYYYGMDV

SEQ ID NO: 49 - protein, 7 aa, synthetic construct
GFTFSSY

SEQ ID NO: 50 - protein, 6 aa, synthetic construct
SYDGSN

SEQ ID NO: 51 - protein, 21 aa, synthetic construct
APYYGSGSYYNSYYYYHGMDV

SEQ ID NO: 52 - protein, 7 aa, synthetic construct
GFTFSNA

SEQ ID NO: 53 - protein, 8 aa, synthetic construct
KSKTDGGT

SEQ ID NO: 54 - protein, 17 aa, synthetic construct
DPRIAVTGTDYYYGMDV

SEQ ID NO: 55 - protein, 11 aa, synthetic construct
RASQSVSSYLA

SEQ ID NO: 56 - protein, 7 aa, synthetic construct
DASNRAT

SEQ ID NO: 57 - protein, 9 aa, synthetic construct
QQRSNWPFT

SEQ ID NO: 58 - protein, 11 aa, synthetic construct
RASQSFSSYLA

SEQ ID NO: 59 - protein, 7 aa, synthetic construct
DTSNRAT

SEQ ID NO: 60 - protein, 9 aa, synthetic construct
QQRSNWPLT

SEQ ID NO: 61 - protein, 11 aa, synthetic construct
RASQGISNYLV

SEQ ID NO: 62 - protein, 7 aa, synthetic construct
AASILQS

SEQ ID NO: 63 - protein, 9 aa, synthetic construct
QQYNTYPFT

SEQ ID NO: 64 - protein, 11 aa, synthetic construct
RASQSVSSYLA

SEQ ID NO: 65 - protein, 7 aa, synthetic construct
DASNRAT

SEQ ID NO: 66 - protein, 10 aa, synthetic construct
QQRSDWPPLT

SEQ ID NO: 67 - protein, 11 aa, synthetic construct
RASQDINNYLA

SEQ ID NO: 68 - protein, 7 aa, synthetic construct
AVSSLES

SEQ ID NO: 69 - protein, 9 aa, synthetic construct
QQYNSYPFT

SEQ ID NO: 70 - protein, 11 aa, synthetic construct
QASQDIINYLN

SEQ ID NO: 71 - protein, 7 aa, synthetic construct
DTSNLET

SEQ ID NO: 72 - protein, 9 aa, synthetic construct
QQFVNLPLT

SEQ ID NO: 73 - protein, 11 aa, synthetic construct
QASQDISNYLN

SEQ ID NO: 74 - protein, 7 aa, synthetic construct
DASNLET

SEQ ID NO: 75 - protein, 9 aa, synthetic construct
QQYDNLPLT

SEQ ID NO: 76 - protein, 11 aa, synthetic construct
QASQDISNYLN

SEQ ID NO: 77 - protein, 7 aa, synthetic construct
DASNLET

SEQ ID NO: 78 - protein, 9 aa, synthetic construct
QQYDNLPLT

SEQ ID NO: 79 - protein, 11 aa, synthetic construct
RASQDISNYLN

SEQ ID NO: 80 - protein, 7 aa, synthetic construct
DASNLET

SEQ ID NO: 81 - protein, 9 aa, synthetic construct
QQYDNLPIT

SEQ ID NO: 82 - protein, 127 aa, synthetic construct
EVQMVESGGDVVRPGGSLRLSCAASGFMFDDYGMSWVRHVQGKGLEWVSGIDWNGDSTGYADSVKGRFTISRDNAKNSLYLQMNSLRAEDTALYYCARDQGIAVAGPYNYYYGMDVWGQGTTVTVSS

SEQ ID NO: 83 - protein, 128 aa, synthetic construct
QVQLVESGGGVVRPGGSLRLSCTASGFTFDDYGMTWVRQAPGKGLEWVSGINWNGGSTGYADSVKGRFTISRDNAKNSLFLQMNSLRAEDTALYYCARDQGIAVVGTHYYYYYGMDVWGQGTAVTVSS

SEQ ID NO: 84 - protein, 117 aa, synthetic construct
QVQLQESGPGMVKPSETLSLTCTVSGGSISYNYWSWIRQPPGKGLEWIGYIYHSGTTKYNPSLKSRVTMSVDTSKNHFSLKLNSVTAADTAVYYCARHEDYWFFDLWGRGTLVTVSS

SEQ ID NO: 85 - protein, 128 aa, synthetic construct
EVQLVESGGGVVRPGGSLRLSCAASGFTFDDSGMSWVRQGPGKGLEWVSGINWNGGSTGYADSVKGRFTISRDNAKNSLYLQMNSLRAEDTALYYCARDPGIAVAGPYYYYYYGMDVWGQGTTVTVSS

SEQ ID NO: 86 - protein, 117 aa, synthetic construct
QVQLQESGPGLVKPSETLSLTCTVSGGSISNYYWSWIRQPPGKGLEWIGYIYYSGSTNYNPSLKSRVNISVDTSKSQVSLKLNSVIAADTAVYYCARHEDYWYFDLWGRGTLVTVSS

SEQ ID NO: 87 - protein, 125 aa, synthetic construct
EVQLVESGGGVVQPGGSLRLSCAASGFTFDDYAMHWVRQAPGKGLEWVSLISGDDGSTYYADSMKGRITISRDNSKNSLYLQMNSLRTEDTAMYYCAKASYGSGSYLYNYGMDVWGQGTTVTVFS

SEQ ID NO: 88 - protein, 128 aa, synthetic construct
QVQLVESGGGLVKPGGSLRLSCAASGFTFSNAWMNWVRQTPGKGLEWVGRIKSKTDGGTKDYAAPVKGRFTISRDDSKNTLYLQMNSLNTEDTAVYYCSTDPRIAVTGTDYYYGMDVWGQGTTVTVSS

SEQ ID NO: 89 - protein, 130 aa, synthetic construct
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAPYYGSGSYYNSYYYYHGMDVWGQGTTVTVSS

SEQ ID NO: 90 - protein, 128 aa, synthetic construct
EVQLVESGGGLVKPGGSLRLSCAASGFTFSNAWMNWVRQAPGKGLEWVGRIKSKTDGGTTDYAAPVKGRFTISRHDSKNTLYLQMNSLKTEDTAVYYCATDPRIAVTGTDYYYGMDVWGQGTTVTVSS

SEQ ID NO: 91 - protein, 107 aa, synthetic construct
EIVLTQSPATLSLSPGERATLSCRASQSVSSYLAWYQEKPGQAPRLLIFDASNRATGIPARFSGSGSGTDFTLTISSLEPEDFAVYYCQQRSNWPFTFGPGTRVEIK

SEQ ID NO: 92 - protein, 107 aa, synthetic construct
EIVLTQSPATLSLSPGERATLSCRASQSFSSYLAWYQQKPGQAPRLLIFDTSNRATGIPARFSGSGSGTDFTLTISSLEPEDFAVYYCQQRSNWPLTFGGGTKVEIK

SEQ ID NO: 93 - protein, 107 aa, synthetic construct
DIQMTQSPSSLSASVGDRVTITCRASQGISNYLVWFQQKPGKAPKSLIYAASILQSGVPSKFSGSGSGTDFTLTINSLQPEDFATYYCQQYNTYPFTFGPGTKVDIK

SEQ ID NO: 94 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSDWPPLTFGGGTKVEIK

SEQ ID NO: 95 - protein, 107 aa, synthetic construct
DIQLTQSPSSLSASIRDRVTITCRASQDINNYLAWFQQKPGKAPKSLIYAVSSLESGVPSKFSGSGSGTDFTLTISSLQPEDFATYFCQQYNSYPFTFGPGTKVEIK

SEQ ID NO: 96 - protein, 107 aa, synthetic construct
DIQLTQSPSSLSASVGDRVTITCQASQDIINYLNWYQQKPGKAPKLLIYDTSNLETGVPSRFSGSGSGTDFAFTINSLQPEDIATYYCQQFVNLPLTFGGGTKVEIK

SEQ ID NO: 97 - protein, 107 aa, synthetic construct
DIQLTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 98 - protein, 107 aa, synthetic construct
AIWMTQSPSSLSASVGDRVTITCQASQDISNYLNWYQQKPGKAPNLLIYDASNLETGVPSRFSGSGSGTDFTLTISSLQPEDIATYYCQQYDNLPLTFGGGTKVEIK

SEQ ID NO: 99 - protein, 107 aa, synthetic construct
DIVMTQTPSFLSASVGDRVTITCRASQDISNYLNWYQQKPGKAPKLLIYDASNLETGVPSRFSGSGSGTDFTFTISSLQPEDIATYYCQQYDNLPITFGQGTRLEIK

SEQ ID NO: 100 - protein, 9 aa, synthetic construct
RMFPNAPYL

SEQ ID NO: 101 - protein, 9 aa, synthetic construct
AMFPNAPYL

SEQ ID NO: 102 - protein, 9 aa, synthetic construct
RMAPNAPYL

SEQ ID NO: 103 - protein, 9 aa, synthetic construct
RMFANAPYL

SEQ ID NO: 104 - protein, 9 aa, synthetic construct
RMFPAAPYL

SEQ ID NO: 105 - protein, 9 aa, synthetic construct
RMFPNAAYL

SEQ ID NO: 106 - protein, 9 aa, synthetic construct
RMFPNAPAL

SEQ ID NO: 107 - protein, 9 aa, synthetic construct
RMFPTPPSL

SEQ ID NO: 108 - protein, 9 aa, synthetic construct
RMFPGEVAL

SEQ ID NO: 109 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 110 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 111 - protein, 121 aa, synthetic construct
QMQLVQSGAEVKEPGESLRISCKGSGYSFTNFWISWVRQMPGKGLEWMGRVDPGYSYSTYSPSFQGHVTISADKSTSTAYLQWNSLKASDTAMYYCARVQYSGYYDWFDPWGQGTLVTVSS

SEQ ID NO: 112 - protein, 111 aa, synthetic construct
QAVVTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKLTVLG

SEQ ID NO: 113 - protein, 124 aa, synthetic construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTQVTVSS

SEQ ID NO: 114 - protein, 107 aa, synthetic construct [sequence omitted]

SEQ ID NO: 115 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 117 - protein, 120 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 118 - protein, 122 aa, synthetic construct [sequence omitted]

SEQ ID NO: 119 - protein, 106 aa, synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 120 - protein, 107 aa, synthetic construct [sequence omitted]

(19) United States
(12) Patent Application Publication
(10) Pub. No.: US 2026/0152571 A1
(43) Pub. Date: Jun. 4, 2026

(54) ANTI-WT1/HLA ANTIBODIES AND USES THEREOF

(71) Applicant: Biocytogen Pharmaceuticals (Beijing) Co., Ltd., Beijing (CN)

(72) Inventors: Jun Du, Beijing (CN); Taolin Liu, Beijing (CN); Limin Zhao, Beijing (CN); Wanbo Tang, Beijing (CN); Ruixue Wang, Beijing (CN)

(21) Appl. No.: 19/123,111

(22) PCT Filed: Oct. 27, 2023
(86) PCT No.: PCT/CN2023/127080

§ 371 Date: Apr. 22, 2025

(30) Foreign Application Priority Data
Oct. 28, 2022 (WO) PCT/CN2022/128430

(51) Int. Cl. C07K 16/46

(52) U.S. Cl. CPC C07K 16/32; A61K 40/11; A61K 40/31; A61K 40/421; A61K 40/4243; A61K 47/6803; A61K 47/6849; A61P 35/00; A61P 35/02; C07K 14/70517; C07K 14/70578; C07K 16/2809; C07K 16/2818; C07K 16/2827; C07K 16/2833; C07K 16/2878; C12N 5/0636; A61K 2039/505; C07K 2317/31; C07K 2317/35; C07K 2317/53; C07K 2317/569; C07K 2317/622; C07K 2317/73; C07K 2317/92; C07K 2319/02; C07K 2319/03; C12N 2510/00

(57) ABSTRACT

Provided are antibodies or antigen-binding fragments thereof that binds to a complex comprising a WT1 peptide and a MHC molecule, and the related antibody-drug conjugate (ADC) derived therefrom, chimeric antigen receptor (CAR) derived therefrom, and the uses thereof.

### CLAIM OF PRIORITY

This application claims priority to PCT/CN2022/128430, filed on Oct. 28, 2022. The entire contents of the foregoing application are incorporated herein by reference.

### TECHNICAL FIELD

This disclosure relates to antibodies or antigen-binding fragments thereof that binds to a complex comprising a WT1 peptide and a MHC molecule, and the related antibody-drug conjugate (ADC) derived therefrom, chimeric antigen receptor (CAR) derived therefrom, and the uses thereof.

### BACKGROUND

WT1 (Wilms tumor 1, Wilms tumor protein) is an oncogenic transcription factor involved in cell proliferation, differentiation, as well as apoptosis and organ development, whose expression in normal adult tissue is rare. WT1 is, however, reported to be overexpressed in several types of haematological maligancies and a wide range of solid tumors. WT1 is a nuclear protein, localized intracellularly. Intracellular protein can be degraded in the proteasome, processed and presented on the cell surface by major histocompatibility complex (MHC) I as T cell epitopes, and recognized by T cell receptors (TCR). As such, WT1-derived peptides can be presented in the context of a human leukocyte antigen (HLA) (specifically, HLA-A2) on the cell surface and can trigger T cell recognition.

Considering the important role of WT1 in tumors, there is a need to develop a therapeutic agent targeting WT1.

### SUMMARY

This disclosure relates to anti-WT1/HLA antibodies, antigen-binding fragment thereof, antibody-drug conjugate (ADC) derived therefrom, chimeric antigen receptor (CAR) derived therefrom, and the uses thereof.

In one aspect, the disclosure is related to an antibody or antigen-binding fragment thereof that binds to a complex comprising a WT1 peptide and a MHC molecule, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
the selected VH CDRs 1, 2, 3 amino acid sequences are set forth in SEQ ID NOs: 1-3, respectively, and the selected VL CDRs 1, 2, 3 amino acid sequences are set forth in SEQ ID NOs: 55-57, respectively;
the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 58-60, respectively;
the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 61-63, respectively;
the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 64-66, respectively;
the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 67-69, respectively;
the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 70-72, respectively;
the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 73-75, respectively;
the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 76-78, respectively;
the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 79-81, respectively;
the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 55-57, respectively;
the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 58-60, respectively;
the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 61-63, respectively;
the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 64-66, respectively;
the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 67-69, respectively;
the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 70-72, respectively;
the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 73-75, respectively;
the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 76-78, respectively; and the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 79-81, respectively.

In some embodiments, the VH comprises CDRs 1, 2, 3 with the amino acid sequences set forth in SEQ ID NOs: 1-3, respectively, and the VL comprises CDRs 1, 2, 3 with the amino acid sequences set forth in SEQ ID NOs: 55-57, respectively, according to Kabat definition.

In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 58-60, respectively, according to Kabat definition.

In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 61-63, respectively, according to Kabat definition.

In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 64-66, respectively, according to Kabat definition.

In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 67-69, respectively, according to Kabat definition.

In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 70-72, respectively, according to Kabat definition.

In some embodiments, the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 73-75, respectively, according to Kabat definition.

In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 76-78, respectively, according to Kabat definition.

In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 79-81, respectively, according to Kabat definition.

In some embodiments, the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 55-57, respectively, according to Chothia definition.

In some embodiments, the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 58-60, respectively, according to Chothia definition.

In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 61-63, respectively, according to Chothia definition.

In some embodiments, the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 64-66, respectively, according to Chothia definition.

In some embodiments, the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 67-69, respectively, according to Chothia definition.

In some embodiments, the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 70-72, respectively, according to Chothia definition.

In some embodiments, the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 73-75, respectively, according to Chothia definition.

In some embodiments, the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 76-78, respectively, according to Chothia definition.

In some embodiments, the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 79-81, respectively, according to Chothia definition.

In some embodiments, the antibody or antigen-binding fragment thereof specifically binds to a complex comprising a human WT1 peptide and a MHC molecule.

In some embodiments, the WT1 peptide comprises a sequence that is at least 80%, 85%, 90%, 95%, or 100% identical to the amino acid sequence of SEQ ID NO: 100.

In some embodiments, the MHC is HLA (e.g., HLA-A2).

In some embodiments, the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure is related to a nucleic acid comprising a polynucleotide encoding a polypeptide comprising:
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1-3, respectively, and wherein the VH, when paired with a light chain variable region (VL) comprising the amino acid sequence set forth in SEQ ID NO: 91 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 55-57, respectively, and wherein the VL, when paired with a VH comprising the amino acid sequence set forth in SEQ ID NO: 82 binds to a complex comprising a WT1 peptide and a MHC molecule;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 92 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 58-60, respectively, and wherein the VL, when paired with a VH comprising the amino acid sequence set forth in SEQ ID NO: 83 binds to a complex comprising a WT1 peptide and a MHC molecule;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 93 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 61-63, respectively, and wherein the VL, when paired with a VH comprising the amino acid sequence set forth in SEQ ID NO: 84 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 94 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 85 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 95 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 67-69, respectively, and wherein the VL, when paired with a VH comprising the amino acid sequence set forth in SEQ ID NO: 86 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 96 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 70-72, respectively, and wherein the VL, when paired with a VH comprising the amino acid sequence set forth in SEQ ID NO: 87 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 97 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 73-75, respectively, and wherein the VL, when paired with a VH comprising the amino acid sequence set forth in SEQ ID NO: 88 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 98 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 76-78, respectively, and wherein the VL, when paired with a VH comprising the amino acid sequence set forth in SEQ ID NO: 89 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 99 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 79-81, respectively, and wherein the VL, when paired with a VH comprising the amino acid sequence set forth in SEQ ID NO: 90 binds to a complex comprising a WT1 peptide and a MHC molecule;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 91 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 55-57, respectively, and wherein the VL, when paired with a VH comprising the amino acid sequence set forth in SEQ ID NO: 82 binds to a complex comprising a WT1 peptide and a MHC molecule;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 92 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 58-60, respectively, and wherein the VL, when paired with a VH comprising the amino acid sequence set forth in SEQ ID NO: 83 binds to a complex comprising a WT1 peptide and a MHC molecule;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 93 binds to a complex comprising a WT1 peptide and a MHC molecule;
an immunoglobulin light chain or a fragment thereof comprising a VL comprising complementarity determining regions (CDRs) 1, 2, and 3 comprising the amino acid sequences set forth in SEQ ID NOs: 61-63, respectively, and wherein the VL, when paired with a VH comprising the amino acid sequence set forth in SEQ ID NO: 84 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 94 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 85 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 40-42, respectively, and wherein the VH, when paired with a light chain variable region (VL) comprising the amino acid sequence set forth in SEQ ID NO: 95 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 67-69, respectively, and wherein the VL, when paired with a VH comprising the amino acid sequence set forth in SEQ ID NO: 86 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 96 binds to a MHC complex comprising a WT1 peptide;
an immunoglobulin light chain or a fragment thereof comprising a VL comprising CDRs 1, 2, and 3 comprising the amino acid sequences set forth in SEQ ID NOs: 70-72, respectively, and wherein the VL, when paired with a VH comprising the amino acid sequence set forth in SEQ ID NO: 87 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 46-48, respectively, and wherein the VH, when paired with a light chain variable region (VL) comprising the amino acid sequence set forth in SEQ ID NO: 97 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 73-75, respectively, and wherein the VL, when paired with a VH comprising the amino acid sequence set forth in SEQ ID NO: 88 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 49-51, respectively, and wherein the VH, when paired with a light chain variable region (VL) comprising the amino acid sequence set forth in SEQ ID NO: 98 binds to a complex comprising a WT1 peptide and a MHC molecule;
an immunoglobulin light chain or a fragment thereof comprising a VL comprising CDRs 1, 2, and 3 comprising the amino acid sequences set forth in SEQ ID NOs: 76-78, respectively, and wherein the VL, when paired with a VH comprising the amino acid sequence set forth in SEQ ID NO: 89 binds to a complex comprising a WT1 peptide and a MHC molecule;
an immunoglobulin heavy chain or a fragment thereof comprising a VH comprising complementarity determining regions (CDRs) 1, 2, and 3 comprising the amino acid sequences set forth in SEQ ID NOs: 52-54, respectively, and wherein the VH, when paired with a light chain variable region (VL) comprising the amino acid sequence set forth in SEQ ID NO: 99 binds to a complex comprising a WT1 peptide and a MHC molecule; or
an immunoglobulin light chain or a fragment thereof comprising a VL comprising CDRs 1, 2, and 3 comprising the amino acid sequences set forth in SEQ ID NOs: 79-81, respectively, and wherein the VL, when paired with a VH comprising the amino acid sequence set forth in SEQ ID NO: 90 binds to a complex comprising a WT1 peptide and a MHC molecule.

In some embodiments, the VH when paired with a VL specifically binds to a complex comprising a WT1 peptide and a MHC molecule, or the VL when paired with a VH specifically binds to a complex comprising a WT1 peptide and a MHC molecule.

In some embodiments, the immunoglobulin heavy chain or the fragment thereof is a human immunoglobulin heavy chain or a fragment thereof (e.g., a human IgG1 heavy chain or a fragment thereof, a human IgG2 heavy chain or a fragment thereof, or a human IgG4 heavy chain or a fragment thereof), and the immunoglobulin light chain or the fragment thereof is a human immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VL region and the VH region that together bind to a complex comprising a WT1 peptide and a MHC molecule.

In one aspect, the disclosure is related to a pair of vectors, wherein each vector comprises one of the nucleic acids described herein, wherein together the pair of vectors encodes the VL region and the VH region that together bind to a complex comprising a WT1 peptide and a MHC molecule.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a cell comprising two of the nucleic acids described herein.

In some embodiments, the two nucleic acids together encode the VL region and the VH region that together bind to a complex comprising a human WT1 peptide and a MHC molecule.

In one aspect, the disclosure is related to a method of producing an antibody or an antigen-binding fragment thereof, the method comprising culturing the cell described herein under conditions sufficient for the cell to produce the antibody or the antigen-binding fragment; and collecting the antibody or the antigen-binding fragment produced by the cell.

In one aspect, the disclosure is related to an antibody or antigen-binding fragment thereof that binds to a complex comprising a WT1 peptide and a MHC molecule, comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
the selected VH sequence is SEQ ID NO: 82, and the selected VL sequence is SEQ ID NO: 91;
the selected VH sequence is SEQ ID NO: 83, and the selected VL sequence is SEQ ID NO: 92;
the selected VH sequence is SEQ ID NO: 84, and the selected VL sequence is SEQ ID NO: 93;
the selected VH sequence is SEQ ID NO: 85, and the selected VL sequence is SEQ ID NO: 94;
the selected VH sequence is SEQ ID NO: 86, and the selected VL sequence is SEQ ID NO: 95;
the selected VH sequence is SEQ ID NO: 87, and the selected VL sequence is SEQ ID NO: 96;
the selected VH sequence is SEQ ID NO: 88, and the selected VL sequence is SEQ ID NO: 97;
the selected VH sequence is SEQ ID NO: 89, and the selected VL sequence is SEQ ID NO: 98; and
the selected VH sequence is SEQ ID NO: 90, and the selected VL sequence is SEQ ID NO: 99.

In some embodiments, the VH comprises the sequence of SEQ ID NO: 82 and the VL comprises the sequence of SEQ ID NO: 91.

In some embodiments, the VH comprises the sequence of SEQ ID NO: 83 and the VL comprises the sequence of SEQ ID NO: 92.

In some embodiments, the VH comprises the sequence of SEQ ID NO: 84 and the VL comprises the sequence of SEQ ID NO: 93.

In some embodiments, the VH comprises the sequence of SEQ ID NO: 85 and the VL comprises the sequence of SEQ ID NO: 94.

In some embodiments, the VH comprises the sequence of SEQ ID NO: 86 and the VL comprises the sequence of SEQ ID NO: 95.

In some embodiments, the VH comprises the sequence of SEQ ID NO: 87 and the VL comprises the sequence of SEQ ID NO: 96.

In some embodiments, the VH comprises the sequence of SEQ ID NO: 88 and the VL comprises the sequence of SEQ ID NO: 97.

In some embodiments, the VH comprises the sequence of SEQ ID NO: 89 and the VL comprises the sequence of SEQ ID NO: 98.

In some embodiments, the VH comprises the sequence of SEQ ID NO: 90 and the VL comprises the sequence of SEQ ID NO: 99.

In one aspect, the disclosure is related to an antibody or antigen-binding fragment thereof that binds to a complex comprising a WT1 peptide and a MHC molecule, comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
the selected VH sequence is SEQ ID NO: 82, and the selected VL sequence is SEQ ID NO: 91;
the selected VH sequence is SEQ ID NO: 83, and the selected VL sequence is SEQ ID NO: 92;
the selected VH sequence is SEQ ID NO: 84, and the selected VL sequence is SEQ ID NO: 93;
the selected VH sequence is SEQ ID NO: 85, and the selected VL sequence is SEQ ID NO: 94;
the selected VH sequence is SEQ ID NO: 86, and the selected VL sequence is SEQ ID NO: 95;
the selected VH sequence is SEQ ID NO: 87, and the selected VL sequence is SEQ ID NO: 96;
the selected VH sequence is SEQ ID NO: 88, and the selected VL sequence is SEQ ID NO: 97;
the selected VH sequence is SEQ ID NO: 89, and the selected VL sequence is SEQ ID NO: 98; and
the selected VH sequence is SEQ ID NO: 90, and the selected VL sequence is SEQ ID NO: 99.

In some embodiments, the antibody or antigen-binding fragment thereof specifically binds to a complex comprising a WT1 peptide and a MHC molecule.

In some embodiments, the WT1 peptide comprises a sequence that is at least 80%, 85%, 90%, 95%, or 100% identical to the amino acid sequence of SEQ ID NO: 100.

In some embodiments, the MHC is HLA (e.g., HLA-A2).

In some embodiments, the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

In some embodiments, the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In one aspect, the disclosure is related to a protein construct that binds to a complex comprising a WT1 peptide and a MHC molecule, comprising:
(1) a first functional moiety comprising an antigen-binding fragment thereof described herein; and
(2) a second functional moiety comprising a T-cell engaging molecule.

In some embodiments, the T-cell engaging molecule (e.g., scFv or VHH) targets human CD3.

In some embodiments, the WT1 peptide comprises a sequence that is at least 80%, 85%, 90%, 95%, or 100% identical to the amino acid sequence of SEQ ID NO: 100.

In some embodiments, the MHC molecule is HLA (e.g., HLA-A2).

In some embodiments, the first functional moiety and the second functional moiety are connected via a linker.

In one aspect, the disclosure is related to a protein construct, comprising:
(1) a first functional moiety comprising an antibody or antigen-binding fragment thereof described herein; and
(2) a second functional moiety comprising a T-cell engaging molecule; and
(3) a third functional moiety comprising a single chain human crystalizable fragment.

In some embodiments, the T-cell engaging molecule is a scFv or VHH targeting human CD3.

In some embodiments, the first functional moiety, the second functional moiety, and the third functional moiety are connected via one or more linkers.

In some embodiments, the WT1 peptide comprises a sequence that is at least 80%, 85%, 90%, 95%, or 100% identical to the amino acid sequence of SEQ ID NO: 100.

In some embodiments, the MHC molecule is HLA (e.g., HLA-A2).

In one aspect, the disclosure is related to an antibody-drug conjugate comprising the antibody or antigen-binding fragment thereof described herein or the protein construct described herein covalently bound to a therapeutic agent.

In some embodiments, the therapeutic agent is a cytotoxic or cytostatic agent.

In one aspect, the disclosure is related to an engineered receptor comprising the antigen-binding fragment thereof described herein.

In some embodiments, the engineered receptor further comprises a transmembrane region, and an intracellular signaling domain.

In some embodiments, the engineered receptor is a chimeric antigen receptor (“CAR”).

In some embodiments, the engineered receptor further comprises a hinge region.

In some embodiments, the transmembrane region comprises a transmembrane region of CD4, CD8, and/or CD28, or a portion thereof.

In some embodiments, the intracellular signaling domain comprises a primary intracellular signaling sequence of an immune effector cell.

In some embodiments, the intracellular signaling domain is or comprises a functional signaling domain of CD3 zeta.

In some embodiments, the intracellular signaling domain further comprises a costimulatory signaling domain.

In some embodiments, the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D). CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand.

In some embodiments, the costimulatory signaling domain comprises an intracellular signaling domain of 4-1BB and/or CD28.

In some embodiments, the engineered receptor comprises a signal peptide.

In some embodiments, the engineered receptor is a chimeric T cell receptor (“cTCR”).

In one aspect, the disclosure is related to a polynucleotide encoding the engineered receptor described herein.

In one aspect, the disclosure is related to a vector comprising the polynucleotide described herein.

In some embodiments, the vector is a viral vector.

In one aspect, the disclosure is related to an engineered cell expressing the engineered receptor described herein.

In some embodiments, the engineered cell is an immune cell.

In some embodiments, the immune cell is an NK cell or a T cell.

In some embodiments, the engineered cell is a T cell.

In some embodiments, the T cell is selected from the group consisting of cytotoxic T cell, a helper T cell, a natural killer T (NK-T) cell, and a γδT cell.

In one aspect, the disclosure is related to a method for producing an engineered cell, comprising introducing a vector described herein into a cell in vitro or ex vivo.

In some embodiments, the vector is a viral vector and the introducing is carried out by transduction.

In one aspect, the disclosure is related to a method of treating a subject having cancer, the method comprising administering a therapeutically effective amount of a composition comprising the antibody or antigen-binding fragment thereof described herein, the protein construct described herein, the engineered cell described herein, or the antibody-drug conjugate described herein, to the subject.

In some embodiments, the subject has a solid tumor.

In some embodiments, the cancer is leukemia, breast cancer, ovarian cancer, glioblastoma or soft tissue sarcoma.

In some embodiments, the subject is further treated with an effective amount of an anti-4-1BB antibody, an anti-OX40 antibody, an anti-CD40 antibody, anti-PD-1 antibody, anti-CTLA4 antibody, or an anti-PD-L1 antibody.

In one aspect, the disclosure is related to a method of decreasing the rate of tumor growth, the method comprising contacting a tumor cell with an effective amount of a composition comprising an antibody or antigen-binding fragment thereof described herein, the protein construct described herein, the engineered cell described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the protein construct described herein, the engineered cell described herein, or the antibody-drug conjugate described herein.

In one aspect, the disclosure is related to a method of increasing immune response in a subject, the method comprising administering to the subject an effective amount of a composition comprising the antibody or antigen-binding fragment thereof described herein, the protein construct described herein, the engineered cell described herein, or the antibody-drug conjugate described herein.

In one aspect, the disclosure is related to a pharmaceutical composition comprising the antibody or antigen-binding fragment thereof described herein, the protein construct described herein, the engineered cell described herein, or the antibody-drug conjugate described herein, and a pharmaceutically acceptable carrier.

In some embodiments, the drug-to-antibody ratio (DAR) of the antibody-drug conjugate described herein is about 4.

In some embodiments, the antibody or antigen-binding fragment thereof described herein binds to positions 2 and/or 9 of the WT1 peptide in the complex comprising a WT1 peptide and a MHC molecule.

In some embodiments, the antibody or antigen-binding fragment thereof described herein binds to positions 1, 3, 4, 5, and/or 8 of the WT1 peptide in the complex comprising a WT1 peptide and a MHC molecule.

In one aspect, the disclosure is related to an antibody or antigen-binding fragment thereof that binds to a complex comprising a WT1 peptide and a MHC molecule, wherein the antibody or antigen-binding fragment thereof binds specifically to:
(1) an amino acid residue corresponding to position 2 of the WT1 peptide in the complex comprising a WT1 peptide and a MHC molecule,
(2) an amino acid residue corresponding to position 9 of the WT1 peptide in the complex comprising a WT1 peptide and a MHC molecule,
(3) an amino acid residue corresponding to position 1 of the WT1 peptide in the complex comprising a WT1 peptide and a MHC molecule,
(4) an amino acid residue corresponding to position 3 of the WT1 peptide in the complex comprising a WT1 peptide and a MHC molecule,
(5) an amino acid residue corresponding to position 4 of the WT1 peptide in the complex comprising a WT1 peptide and a MHC molecule,
(6) an amino acid residue corresponding to position 5 of the WT1 peptide in the complex comprising a WT1 peptide and a MHC molecule, and/or
(7) an amino acid residue corresponding to position 8 of the WT1 peptide in the complex comprising a WT1 peptide and a MHC molecule, wherein the WT1 peptide has the sequence set forth in SEQ ID NO: 100.

In one aspect, the disclosure is related to an antibody or antigen-binding fragment thereof that binds to a complex comprising a WT1 peptide and a MHC molecule, wherein the antibody or antigen-binding fragment thereof binds specifically to an epitope in the WT1 peptide, wherein the epitope is one or more of the following:
(1) an amino acid residue corresponding to R1 of SEQ ID NO: 100;
(2) an amino acid residue corresponding to F3 of SEQ ID NO: 100;
(3) an amino acid residue corresponding to P4 of SEQ ID NO: 100;
(4) an amino acid residue corresponding to N5 of SEQ ID NO: 100; and
(5) an amino acid residue corresponding to Y8 of SEQ ID NO: 100.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoictic neoplastic disorder can arise from myeloid, lymphoid or crythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, single variable domain (V_{H}H) antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “anti-WT1/HLA antibody” refers to an antibody that can specifically binds to a MHC complex comprising a WT1 peptide and a HLA-A2 molecule (also be referred to a complex comprising a WT1 peptide and a MHC molecule).

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a WT1/HLA-A2 complex may be referred to as a WT1/HLA-A2-specific antibody, an anti-WT1/HLA antibody, or an anti-WT1/HLA complex antibody.

As used herein, the term “bispecific antibody” refers to an antibody that binds to two different epitopes. The epitopes can be on the same antigen or on different antigens.

As used herein, the term “multi-specific antibody” refers to an antibody that binds to two or more different epitopes. The epitopes can be on the same antigen or on different antigens. A multispecific antibody can be e.g., a bispecific antibody or a trispecific antibody. In some embodiments, the multi-specific antibody binds to two, three, four, five, or six different epitopes.

As used herein, a “chimeric antigen receptor” or “CAR” refers to a fusion protein comprising an extracellular domain capable of binding to an antigen, and an intracellular region comprising one or more intracellular signaling domains derived from signal transducing proteins. The extracellular domain can be any proteinaceous molecule or part thereof that can specifically bind to a predetermined antigen. In some embodiments, the extracellular domain comprises an antibody or antigen binding fragment thereof. In some embodiments, the intracellular signaling domain can be any oligopeptide or polypeptide domain known to function to transmit a signal causing activation or inhibition of a biological process in a cell, for example, activation of an immune cell such as a T cell or a NK cell.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1C show exemplary antibody structures of the bispecific antibody.

FIG. 1D shows an exemplary structure of a construct.

FIG. 2 lists heavy chain variable region and light chain variable region CDR sequences of anti-WT1/HLA antibodies under Kabat definition.

FIG. 3 lists heavy chain variable region and light chain variable region CDR sequences of anti-WT1/HLA antibodies under Chothia definition.

FIGS. 4A-4J show the binding between T2 cells pulsed with alanine-replaced peptides (25 μM) and the anti-WT1/HLA antibodies, including ESK1 analog (FIG. 4A), 7B9-IgG1-SI (FIG. 4B), P01264-IgG1-SI (FIG. 4C), 1E9-IgG1-SI (FIG. 4D), P01218-IgG1-SI (FIG. 4E), P01199-IgG1-SI (FIG. 4F), 1G7-IgG1-SI (FIG. 4G), 3E6-IgG1-SI (FIG. 4H), 3A6-IgG1-SI (FIG. 4I), and 4F10-IgG1-SI (FIG. 4J), as determined by flow cytometry.

FIGS. 5A-5D show the cytotoxicity induced by anti-WT1/CD3 bispecific antibodies including 1G7-CD3 (FIG. 5A), 7B9-CD3 (FIG. 5B), P01199-CD3 (FIG. 5C), P01218-CD3 (FIG. 5D) in CD3+ T cells against WT1 and HLA-A2 positive tumor cells.

FIG. 6 shows the human IFN-γ cytokine release at 24 h as measured by ELISA.

FIG. 7 shows the average tumor volume in different groups of B-NDG mice that were subcutaneously injected with HCT116-WT1 cells, and were treated with PBS or anti-WT1/CD3 bispecific antibodies.

FIG. 8 shows the average tumor volume in different groups of B-NDG mice that were subcutaneously injected with HCT116-WT1 cells, and were treated with saline, mock T cells or CAR-T cells (1G7-CAR-T (VH-VL), 3E6-CAR-T (VH-VL), P01264-CAR-T (VH-VL), ESK1-CAR-T (VH-VL), 1G7-CAR-T (VL-VH), 3E6-CAR-T (VL-VH), P01264-CAR-T (VL-VH)).

FIG. 9 lists selected amino acid sequences discussed in the disclosure.

FIG. 10 shows the average tumor volume in different groups of B-NDG mice that were subcutaneously injected with HCT116-WT1 cells, and were treated with saline, mock T cells or CAR-T cells (1E9-CAR-T (VL-VH), 3A6-CAR-T (VL-VH), 4F10-CAR-T (VL-VH), P01199-CAR-T (VL-VH), P01218-CAR-T (VL-VH)).

FIG. 11 shows the survival mice rate in different groups of B-NDG mice that were intravenously injected with THP-1-luc cells, and were treated with saline, mock T cells or CAR-T cells (1E9-CAR-T (VL-VH), 3A6-CAR-T (VL-VH), 4F10-CAR-T (VL-VH), P01199-CAR-T (VL-VH), P01218-CAR-T (VL-VH)).

FIG. 12 shows the cytotoxicity induced by anti-WT1/CD3 bispecific antibodies 1E9-CD3, 1G7-CD3, and P01218-CD3 in CD3+ T cells against THP-1 cells or OVCAR3 cells. The RG6007 analog is a positive control.

FIG. 13 shows the cytotoxicity induced by anti-WT1/CD3 bispecific antibodies 3E6-CD3, 7B9-CD3, and P01199-CD3 in CD3+ T cells against THP-1 cells or OVCAR3 cells. The RG6007 analog is a positive control.

FIG. 14 shows the cytotoxicity induced by anti-WT1/CD3 bispecific antibodies 1E9-CD3 (VHH), 1G7-CD3 (VHH), 3A6-CD3 (VHH), 3E6-CD3 (VHH), 4F10-CD3 (VHH), 7B9-CD3 (VHH), P01199-CD3 (VHH), P01218-CD3 (VHH) and P01264-CD3 (VHH) in CD3+ T cells against THP-1 cells or OVCAR3 cells.

FIGS. 15A-15B show the binding between T2 cells pulsed with WT1_{RMF}. MED13L peptide or PIGQ peptide and 1G7-IgG1-SI (FIG. 15A) or RG6007-WT1 (FIG. 15B), as determined by flow cytometry.

### DETAILED DESCRIPTION

The present disclosure provides examples of antibodies, antigen-binding fragment thereof, that bind to a MHC complex comprising a WT1 peptide.

### WT1

The WT1 (Wilms' tumor 1) gene, which encodes a protein consisting of four zinc finger domains at the C terminus and a glutamine and proline-rich domain at the N terminus, plays an important role in cell proliferation, differentiation, apoptosis, organ development and the maintenance of several adult tissues. Though recognized as a classic tumor suppressor gene in Wilms' tumor, there is a growing body of evidence demonstrating that wild-type WT1 is expressed in a variety of tumors arising from different tissues that normally do not express WT1. It is possible that WT1 could inhibit cell apoptosis by transcriptional activation and/or upregulation of proto-oncogenes. Recent work has also revealed that WT1 is a key regulator in overcoming senescence downstream of KRAS signaling, and is involved in the apoptotic response to cytotoxic stress, further demonstrating its oncogenic effects. Moreover, WT1 could also promote invasion, migration and metastasis, facilitate angiogenesis, and confer drug resistance to cancer cells. WT1 is overexpressed in a number of cancer cells. Particularly, WT1 is expressed at high levels in a variety of adult epithelial tumors and in some leukaemias, and this has led to much endeavour in the pursuit of immune therapies targeting WT1 epitopes.

A detailed review of WT1 and its functions can be found in Qi, Xiao-wei, et al. “Wilms' tumor 1 (WT1) expression and prognosis in solid cancer patients: a systematic review and meta-analysis.” Scientific reports 5.1 (2015): 1-9; Hastic, Nicholas D. “Wilms' tumour 1 (WT1) in development, homeostasis and disease.” Development 144.16 (2017): 2862-2872; each of which is incorporated by reference in its entirety.

The present disclosure provides “TCR-like” antibodies targeting WT1 peptide-MHC complexes. The development of these TCR-like antibodies based therapeutics can improve the therapeutic efficacy. Thus, in one aspect, the present disclosure provides methods of treating disorders associated with WT1. In some embodiments, the disorder is a disorder that over expresses WT1. In some embodiments, the disorder is cancer.

Among these peptides presented by cells. WT1 (126-134) peptide (WT1_{RMF}, RMFPNAPYL, SEQ ID NO: 100) is a peptide restricted by HLA-A2. In some embodiments, the antibodies or antigen binding fragments as described herein specifically bind to the WT1/HLA-A2 complex.

### Antibodies and Antigen Binding Fragments

The present disclosure provides anti-WT1/HLA antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibodies comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin. “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68(1-3): 9-16 (October 1997); Morea et al., J Mol Biol. 275(2): 269-94 (January 1998); Chothia et al., Nature 342(6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al. “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting a WT1/HLA-A2 complex and an addition antigen (e.g., human CD3).

### Anti-WT1/HLA Antibodies and Antigen-Binding Fragments

The disclosure provides antibodies and antigen-binding fragments thereof that specifically bind to a MHC complex comprising a WT1 peptide. In some embodiments, the MHC molecule is HLA (e.g., HLA-A2). The antibodies and antigen-binding fragments described herein are capable of binding to a MHC complex comprising a WT1 peptide. In some embodiments, these antibodies can increase immune response.

The disclosure provides e.g., anti-WT1/HLA antibodies 1E9, 1G7, 3A6, 3E6, 4F10, 7B9, P01199, P01218, P01264, the chimeric antibodies thereof, and the human or humanized antibodies thereof.

The CDR sequences for 1E9, and 1E9 derived antibodies (e.g., human or humanized antibodies) include CDRs of the heavy chain variable domain. SEQ ID NOs: 1-3, and CDRs of the light chain variable domain, SEQ ID NOs: 55-57 as defined by Kabat definition. The CDRs can also be defined by Chothia definition. Under the Chothia definition, the CDR sequences of the heavy chain variable domain are set forth in SEQ ID NOs: 28-30 and CDR sequences of the light chain variable domain are set forth in SEQ ID NOs: 55-57.

Similarly, the CDR sequences for 1G7, and 1G7 derived antibodies include CDRs of the heavy chain variable domain. SEQ ID NOs: 4-6, and CDRs of the light chain variable domain, SEQ ID NOs: 58-60, as defined by Kabat definition. Under Chothia definition, the CDR sequences of the heavy chain variable domain are set forth in SEQ ID NOs: 31-33, and CDRs of the light chain variable domain are set forth in SEQ ID NOs: 58-60.

The CDR sequences for 3A6, and 3A6 derived antibodies include CDRs of the heavy chain variable domain, SEQ ID NOs: 7-9, and CDRs of the light chain variable domain, SEQ ID NOs: 61-63, as defined by Kabat definition. Under Chothia definition, the CDR sequences of the heavy chain variable domain are set forth in SEQ ID NOs: 34-36, and CDRs of the light chain variable domain are set forth in SEQ ID NOs: 61-63.

The CDR sequences for 3E6, and 3E6 derived antibodies include CDRs of the heavy chain variable domain, SEQ ID NOs: 10-12, and CDRs of the light chain variable domain, SEQ ID NOs: 64-66, as defined by Kabat definition. Under Chothia definition, the CDR sequences of the heavy chain variable domain are set forth in SEQ ID NOs: 37-39, and CDRs of the light chain variable domain are set forth in SEQ ID NOs: 64-66.

The CDR sequences for 4F10, and 4F10 derived antibodies include CDRs of the heavy chain variable domain, SEQ ID NOs: 13-15, and CDRs of the light chain variable domain, SEQ ID NOs: 67-69, as defined by Kabat definition. Under Chothia definition, the CDR sequences of the heavy chain variable domain are set forth in SEQ ID NOs: 40-42, and CDRs of the light chain variable domain are set forth in SEQ ID NOs: 67-69.

The CDR sequences for 7B9, and 7B9 derived antibodies include CDRs of the heavy chain variable domain, SEQ ID NOs: 16-18, and CDRs of the light chain variable domain, SEQ ID NOs: 70-72, as defined by Kabat definition. Under Chothia definition, the CDR sequences of the heavy chain variable domain are set forth in SEQ ID NOs: 43-45, and CDRs of the light chain variable domain are set forth in SEQ ID NOs: 70-72.

The CDR sequences for P01199, and P01199 derived antibodies include CDRs of the heavy chain variable domain, SEQ ID NOs: 19-21, and CDRs of the light chain variable domain, SEQ ID NOs: 73-75, as defined by Kabat definition. Under Chothia definition, the CDR sequences of the heavy chain variable domain are set forth in SEQ ID NOs: 46-48, and CDRs of the light chain variable domain are set forth in SEQ ID NOs: 73-75.

The CDR sequences for P01218, and P01218 derived antibodies include CDRs of the heavy chain variable domain, SEQ ID NOs: 22-24, and CDRs of the light chain variable domain, SEQ ID NOs: 76-78, as defined by Kabat definition. Under Chothia definition, the CDR sequences of the heavy chain variable domain are set forth in SEQ ID NOs: 49-51, and CDRs of the light chain variable domain are set forth in SEQ ID NOs: 76-78.

The CDR sequences for P01264, and P01264 derived antibodies include CDRs of the heavy chain variable domain. SEQ ID NOs: 25-27, and CDRs of the light chain variable domain, SEQ ID NOs: 79-81, as defined by Kabat definition. Under Chothia definition, the CDR sequences of the heavy chain variable domain are set forth in SEQ ID NOs: 52-54, and CDRs of the light chain variable domain are set forth in SEQ ID NOs: 79-81.

The amino acid sequence for the heavy chain variable region of 1E9 antibody is set forth in SEQ ID NO: 82. The amino acid sequence for the light chain variable region of 1E9 antibody is set forth in SEQ ID NO: 91.

The amino acid sequence for the heavy chain variable region of 1G7 antibody is set forth in SEQ ID NO: 83. The amino acid sequence for the light chain variable region of 1G7 antibody is set forth in SEQ ID NO: 92.

The amino acid sequence for the heavy chain variable region of 3A6 antibody is set forth in SEQ ID NO: 84. The amino acid sequence for the light chain variable region of 3A6 antibody is set forth in SEQ ID NO: 93.

The amino acid sequence for the heavy chain variable region of 3F6 antibody is set forth in SEQ ID NO: 85. The amino acid sequence for the light chain variable region of 3E6 antibody is set forth in SEQ ID NO: 94.

The amino acid sequence for the heavy chain variable region of 4F10 antibody is set forth in SEQ ID NO: 86. The amino acid sequence for the light chain variable region of 4F10 antibody is set forth in SEQ ID NO: 95.

The amino acid sequence for the heavy chain variable region of 7B9 antibody is set forth in SEQ ID NO: 87. The amino acid sequence for the light chain variable region of 7B9 antibody is set forth in SEQ ID NO: 96.

The amino acid sequence for the heavy chain variable region of P01199 antibody is set forth in SEQ ID NO: 88. The amino acid sequence for the light chain variable region of P01199 antibody is set forth in SEQ ID NO: 97.

The amino acid sequence for the heavy chain variable region of P01218 antibody is set forth in SEQ ID NO: 89. The amino acid sequence for the light chain variable region of P01218 antibody is set forth in SEQ ID NO: 98.

The amino acid sequence for the heavy chain variable region of P01264 antibody is set forth in SEQ ID NO: 90. The amino acid sequence for the light chain variable region of P01264 antibody is set forth in SEQ ID NO: 99.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to any one of SEQ ID NOs: 82-90. In some embodiments, the light chain variable region is at least 80%, 85%, 90%, 91%, 92%, 93%, 94%, 95%, 96%, 97%, 98%, or 99% identical to any one of SEQ ID NOs: 91-99. The heavy chain variable region sequence can be paired with the corresponding light chain variable region sequence, and together they bind to a WT1/HLA-A2 complex.

Humanization percentage means the percentage identity of the heavy chain or light chain variable region sequence as compared to human antibody sequences in International Immunogenetics Information System (IMGT) database. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, SEQ ID NOS: 37-39, SEQ ID NOs: 40-42, SEQ ID NOs: 43-45, SEQ ID NOs: 46-48, SEQ ID NOs: 49-51, SEQ ID NOs: 52-54; and/or one, two, or three light chain variable region CDRs selected from the group of SEQ ID NOs: 55-57, SEQ ID NOs: 58-60, SEQ ID NOs: 61-63, SEQ ID NOS: 64-66, SEQ ID NOs: 67-69, SEQ ID NOs: 70-72, SEQ ID NOs: 73-75, SEQ ID NOs: 76-78, and SEQ ID NOs: 79-81.

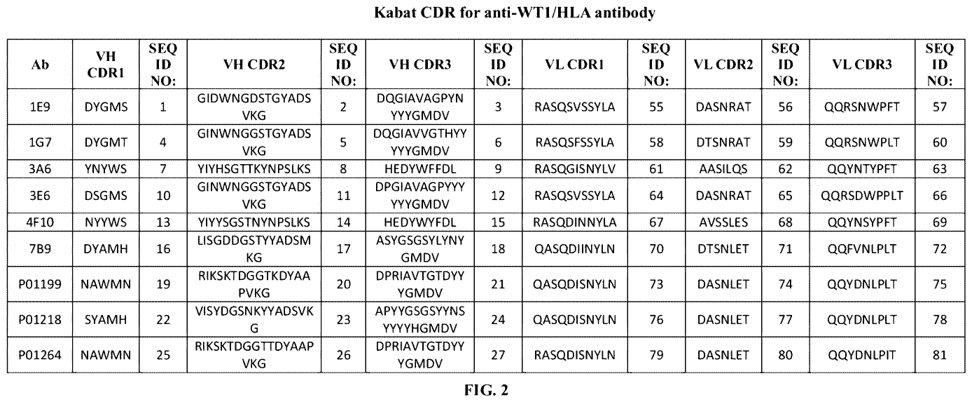

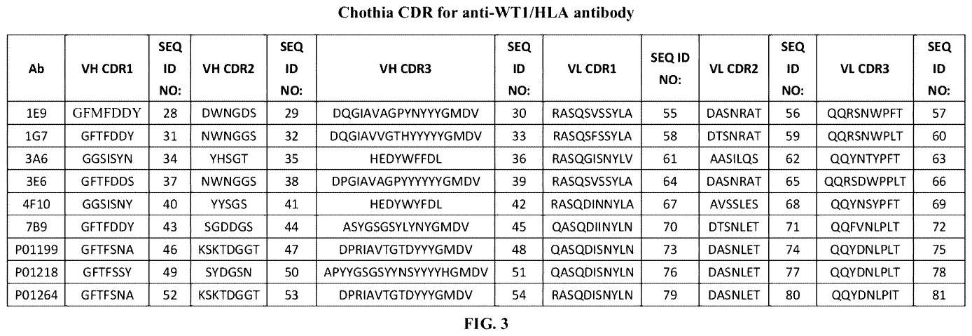

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 2 (Kabat CDR) and FIG. 3 (Chothia CDR).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 52 with zero, one or two amino acid insertions, deletions, or substitutions; SEQ ID NO: 53 with zero, one or two amino acid insertions, deletions, or substitutions; SEQ ID NO: 54 with zero, one or two amino acid insertions, deletions, or substitutions.

In some embodiments, the antibody or an antigen-binding fragment described herein can contain a light chain variable domain containing one, two, or three of the CDRs of SEQ ID NO: 55 with zero, one or two amino acid insertions, deletions, or substitutions; SEQ ID NO: 56 with zero, one or two amino acid insertions, deletions, or substitutions; SEQ ID NO: 57 with zero, one or two amino acid insertions, deletions, or substitutions.

In some embodiments, the antibody or an antigen-binding fragment described herein can contain a light chain variable domain containing one, two, or three of the CDRs of SEQ ID NO: 58 with zero, one or two amino acid insertions, deletions, or substitutions; SEQ ID NO: 59 with zero, one or two amino acid insertions, deletions, or substitutions; SEQ ID NO: 60 with zero, one or two amino acid insertions, deletions, or substitutions.

In some embodiments, the antibody or an antigen-binding fragment described herein can contain a light chain variable domain containing one, two, or three of the CDRs of SEQ ID NO: 61 with zero, one or two amino acid insertions, deletions, or substitutions; SEQ ID NO: 62 with zero, one or two amino acid insertions, deletions, or substitutions; SEQ ID NO: 63 with zero, one or two amino acid insertions, deletions, or substitutions.

In some embodiments, the antibody or an antigen-binding fragment described herein can contain a light chain variable domain containing one, two, or three of the CDRs of SEQ ID NO: 64 with zero, one or two amino acid insertions, deletions, or substitutions; SEQ ID NO: 65 with zero, one or two amino acid insertions, deletions, or substitutions; SEQ ID NO: 66 with zero, one or two amino acid insertions, deletions, or substitutions.

In some embodiments, the antibody or an antigen-binding fragment described herein can contain a light chain variable domain containing one, two, or three of the CDRs of SEQ ID NO: 67 with zero, one or two amino acid insertions, deletions, or substitutions; SEQ ID NO: 68 with zero, one or two amino acid insertions, deletions, or substitutions; SEQ ID NO: 69 with zero, one or two amino acid insertions, deletions, or substitutions.

In some embodiments, the antibody or an antigen-binding fragment described herein can contain a light chain variable domain containing one, two, or three of the CDRs of SEQ ID NO: 70 with zero, one or two amino acid insertions, deletions, or substitutions; SEQ ID NO: 71 with zero, one or two amino acid insertions, deletions, or substitutions; SEQ ID NO: 72 with zero, one or two amino acid insertions, deletions, or substitutions.

In some embodiments, the antibody or an antigen-binding fragment described herein can contain a light chain variable domain containing one, two, or three of the CDRs of SEQ ID NO: 73 with zero, one or two amino acid insertions, deletions, or substitutions; SEQ ID NO: 74 with zero, one or two amino acid insertions, deletions, or substitutions; SEQ ID NO: 75 with zero, one or two amino acid insertions, deletions, or substitutions.

In some embodiments, the antibody or an antigen-binding fragment described herein can contain a light chain variable domain containing one, two, or three of the CDRs of SEQ ID NO: 76 with zero, one or two amino acid insertions, deletions, or substitutions; SEQ ID NO: 77 with zero, one or two amino acid insertions, deletions, or substitutions; SEQ ID NO: 78 with zero, one or two amino acid insertions, deletions, or substitutions.

In some embodiments, the antibody or an antigen-binding fragment described herein can contain a light chain variable domain containing one, two, or three of the CDRs of SEQ ID NO: 79 with zero, one or two amino acid insertions, deletions, or substitutions; SEQ ID NO: 80 with zero, one or two amino acid insertions, deletions, or substitutions; SEQ ID NO: 81 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition. In some embodiments, the CDR is determined based on Chothia definition. In some embodiments, the CDR is determined based on a combination of Kabat definition and Chothia definition.

The disclosure also provides antibodies or antigen-binding fragments thereof that bind to a WT1/HLA-A2 complex. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 82, and the selected VL sequence is SEQ ID NO: 91. In some embodiments, the selected VH sequence is SEQ ID NO: 83 and the selected VL sequence is SEQ ID NO: 92. In some embodiments, the selected VH sequence is SEQ ID NO: 84, and the selected VL sequence is SEQ ID NO: 93. In some embodiments, the selected VH sequence is SEQ ID NO: 85, and the selected VL sequence is SEQ ID NO: 94. In some embodiments, the selected VH sequence is SEQ ID NO: 86, and the selected VL sequence is SEQ ID NO: 95. In some embodiments, the selected VH sequence is SEQ ID NO: 87, and the selected VL sequence is SEQ ID NO: 96. In some embodiments, the selected VH sequence is SEQ ID NO: 88, and the selected VL sequence is SEQ ID NO: 97. In some embodiments, the selected VH sequence is SEQ ID NO: 89, and the selected VL sequence is SEQ ID NO: 98. In some embodiments, the selected VH sequence is SEQ ID NO: 90, and the selected VL sequence is SEQ ID NO: 99.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

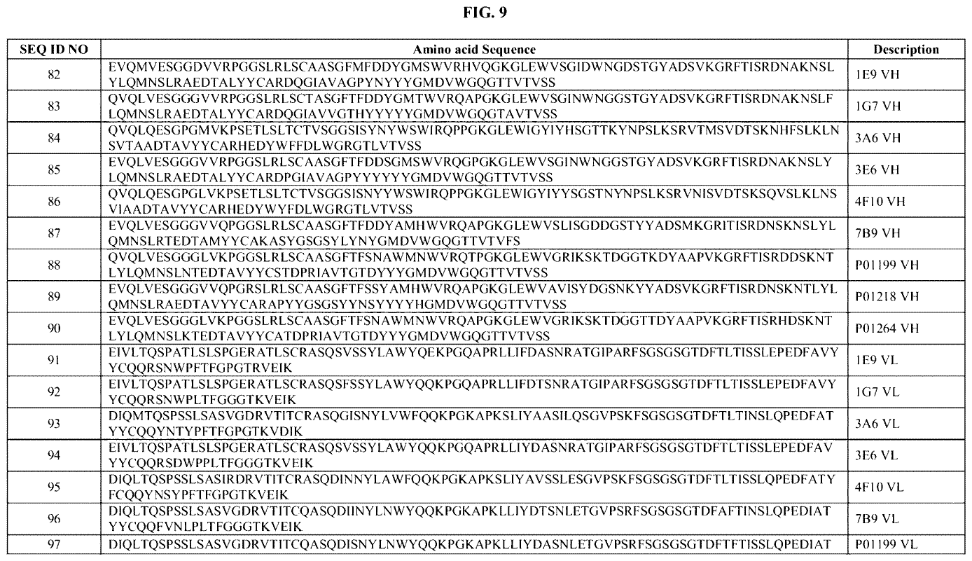

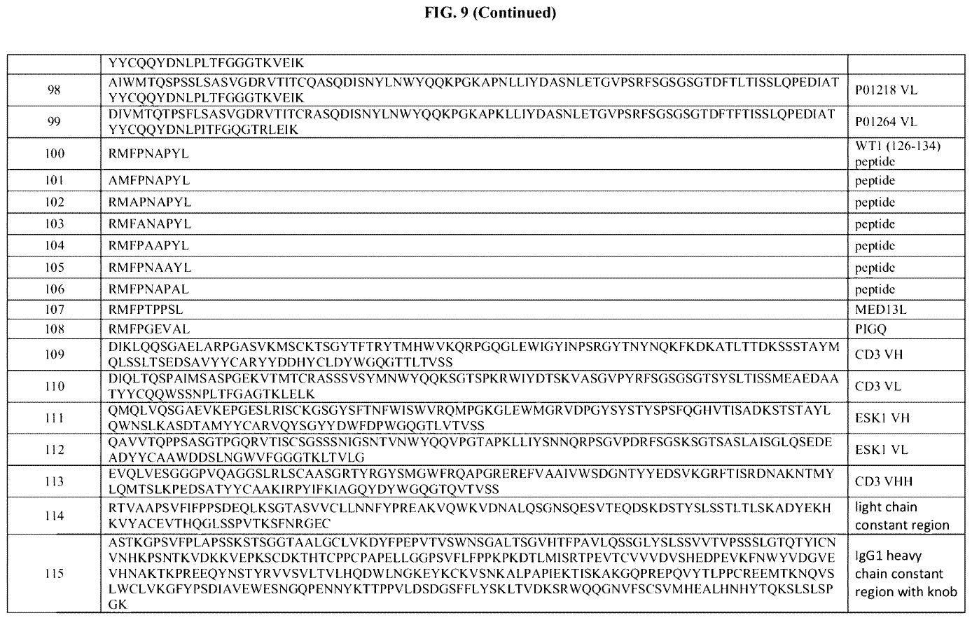

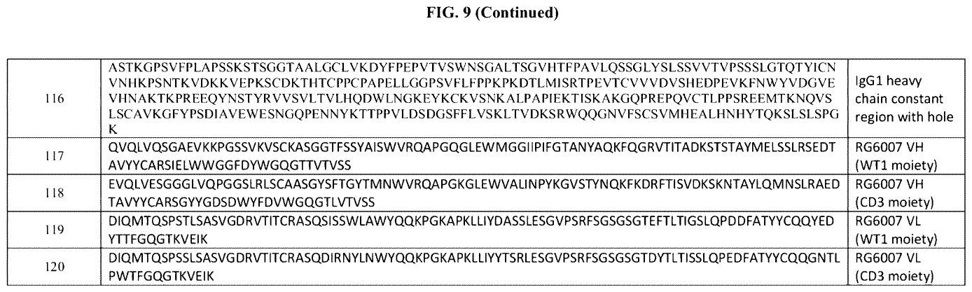

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 2 or FIG. 3, or have sequences as shown in FIG. 9. When the polypeptides are paired with corresponding polypeptide (e.g., a corresponding heavy chain variable region or a corresponding light chain variable region), the paired polypeptides bind to a WT1/HLA-A2 complex.

The anti-WT1/HLA antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2. IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to a WT1/HLA-A2 complex will retain an ability to bind to the WT1/HLA-A2 complex. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

One-armed antibodies can have a heavy chain and a light chain, and a heavy chain fragment comprising CH2 and CH3 domains of IgG. In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

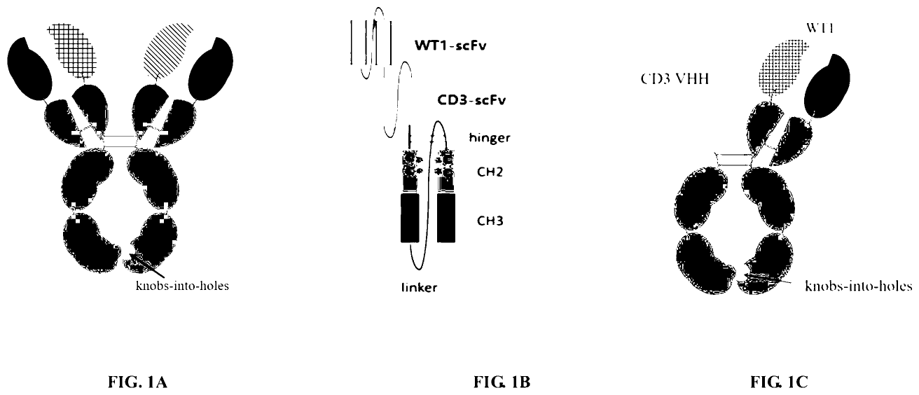

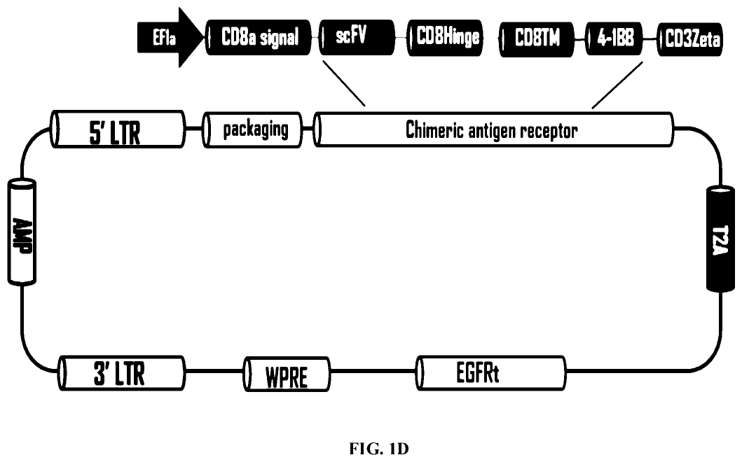

In some embodiments, the disclosure is related to a bispecific antibody that comprises (1) a first functional moiety comprising an antibody or antigen-binding fragment described herein; (2) a second functional moiety comprising a T-cell engaging molecule; and (3) a third functional moiety comprising a single chain human crystalizable fragment. In some embodiments, the T-cell engaging molecule targets CD3. In some embodiments, the T-cell engaging molecule is an anti-CD3 scFv. In some embodiments, the antibody or antigen-binding fragment described herein is a scFv. In some embodiments, the C terminus of the antibody or antigen-binding fragment described herein is linked to the N terminus of an anti-CD3 scFv via a peptide linker, and the C terminus of the anti-CD3 scFv is linked to a single chain human crystalizable fragment via a peptide linker. FIG. 1B illustrates an exemplary structure of a bispecific antibody. In some embodiments, the T-cell engaging molecule is an anti-CD3 VHH. In some embodiments, the antibody or antigen-binding fragment described herein is linked to a human crystalizable fragment via a hinge region, and an anti-CD3 antibody or antigen-binding fragment thereof is linked to the same human crystalizable fragment via a hinge region. FIG. 1C illustrates an exemplary structure of a bispecific antibody.

In some embodiments, provided herein are multi-specific antibodies (e.g., bispecific antibodies) having an anti-WT1 arm and an anti-CD3 arm. In some embodiments, the anti-WT1 arm includes a heavy chain (e.g., any of the heavy chains having a VH described herein), and a light chain (e.g., any of the light chains having a VL described herein). In some embodiments, the anti-CD3 arm includes a heavy chain variable region (VHH) of an anti-CD3 heavy chain antibody. In some embodiments, the VHH comprises or consists of an amino acid sequence that is at least 80%, 85%, 86%, 87%, 88%, 89%, 90%, 91%, 92%, 93%, 94%, 95%, 96%, 97%, 98%, 99% or 100% identical to SEQ ID NO: 113. In some embodiments, the bispecific antibodies described herein have a schematic structure shown in FIG. 1C.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the WT1 peptide comprises a sequence that is at least 80%, 85%, 90%, 95%, or 100% identical to the amino acid sequence of SEQ ID NO: 100.

In some embodiments, the antibodies or antigen-binding fragments described herein does not bind to a MHC molecule without the WT1 peptide.

In some embodiments, the antibodies or antigen-binding fragments described herein does not bind to off-target peptides (sequence-similar peptides derived from protein MED13L and PIGQ), as determined by flow cytometry.

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody). In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the cytotoxic agent is a camptothecin compound, an analogue or a derivative thereof (e.g., compound CPT-1, CPT-2, CPT-3, CPT-4 in patent application PCT/CN2023/093976).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate: defofamine; demecolcine: diaziquone; elfornithine; elliptinium acetate: etoglucid; gallium nitrate; hydroxyurea; lentinan: lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone: vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug. A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative Biol abs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) is about or at least 1, 2, 3, 4, 5, 6, 7, or 8. In some embodiments, the average DAR in the composition is about 1˜about 2, about 2˜about 3, about 3˜about 4, about 3˜about 5, about 4˜about 5, about 5˜about 6, about 6˜about 7, or about 7˜about 8.

### Antibody and ADC Characteristics

In some implementations, the antibody (or antigen-binding fragments thereof) or ADC derived therefrom specifically binds to a WT1/HLA-A2 complex with a dissociation rate (koff) of less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR).

In some embodiments, the binding activity of anti-WT1/HLA antibodies to T2 cells (ATCC, Cat #: CRL-1992) pulsed with WT1_{RMF} were verified by flow cytometry. In some embodiments, the EC50 is less than 0.1 nM, 0.2 nM, 0.4 nM, 0.8 nM, 1 nM, 1.2 nM, 1.4 nM, 1.8 nM, 2 nM, 2.2 nM, 2.4 nM, 2.8 nM, 3 nM, 3.2 nM, 3.4 nM, 3.8 nM, or 4 nM.

ESK1 analog is a fully human TCR-like monoclonal IgG1 antibody targeting the WT1_{RMF}/HLA-A2 complex developed by Eureka Therapeutics, INC (VH SEQ ID NO:111; VL SEQ ID NO:112).

In some embodiments, the key targeting sites of anti-WT1/HLA antibodies on WT1_{RMF} (SEQ ID NO: 100) are different from those of the antibody ESK1 analog.

In some embodiments, the anti-WT1/HLA antibody binds to positions 2 and 9 of the WT1_{RMF} peptide in the WT1_{RMF}/HLA-A2 complex.

In some embodiments, the anti-WT1/HLA antibody binds to positions 1, 3, and 5 of the WT1_{RMF} peptide in the WT1_{RMF}/HLA-A2 complex.

In some embodiments, the anti-WT1/HLA antibody binds to position 1 of the WT1_{RMF} peptide in the WT1_{RMF}/HLA-A2 complex.

In some embodiments, the anti-WT1/HLA antibody binds to positions 1, 3, 4, and 8 of the WT1_{RMF} peptide in the WT1_{RMF}/HLA-A2 complex.

In some embodiments, the anti-WT1/HLA antibody binds to positions 1, 4, and 8 of the WT1_{RMF} peptide in the WT1_{RMF}/HLA-A2 complex.

In some embodiments, the anti-WT1/HLA antibody binds to positions 1, 3, and 4 of the WT1_{RMF} peptide in the WT1_{RMF}/HLA-A2 complex.

In some embodiments, the anti-WT1/HLA antibody binds to positions 3, 4, 5, and 8 of the WT1_{RMF} peptide in the WT1_{RMF}/HLA-A2 complex.

In some embodiments, binding of anti-WT1/HLA antibodies to WT1_{RMF} and potential off-target peptides (sequence-similar peptides derived from protein MED13L and PIGQ) was verified by flow cytometry. In some embodiments, the percentage of positive cells for WT1_{RMF}-pulsed T2 cells is above 90%, above 91%, above 92%, above 93%, above 94%, above 95%, above 96%, above 97%, above 98%, or above 99%. In some embodiments, the percentage of positive cells for T2 cells, MED13L-pulsed T2 cells, or PIGI-pulsed T2 cells is less than 1%, less than 2%, less than 3%, less than 4%, less than 5%, less than 6%, less than 7%, less than 8%, less than 9%, or less than 10%.

In some embodiments, anti-WT1/CD3 bispecific antibodies (1G7-CD3, 7B9-CD3, P01199-CD3, P01218-CD3, P01264-CD3) at different concentrations (0.00001 μg/mL, 0.001 μg/mL, 0.1 μg/mL, 10 μg/mL) were co-incubated with purified CD3 T cells and target cells (HCT116-WT1 cells) at a 10:1 ratio (E:T) for 24 hours. In some embodiments, the IFN-γ cytokine release is above 10 pg/mL, above 25 pg/mL, above 50 pg/mL, above 100 pg/mL, above 250 pg/mL, above 500 pg/mL, above 1000 pg/mL, above 2500 pg/mL, above 5000 pg/mL, or above 7000 pg/mL.

In some embodiments, thermal stabilities are determined. The antibodies or antigen binding fragments as described herein or ADC derived therefrom can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC.

In some embodiments, the antibody or antigen-binding fragment thereof as described herein, the ADC derived therefrom, or the CAR-T cell derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein, ADC derived therefrom, or the CAR-T cell derived therefrom has a tumor growth inhibition percentage that is less than 60%, 70%, 80%, 90%, 100%, 110%. 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100
Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments as described herein or ADC derived therefrom can bind to tumor cells that present a WT1 peptide by a MHC molecule (e.g., HLA or HLA-A2). In some embodiments, the antibodies or antigen binding fragments as described herein or ADC derived therefrom can induce complement-dependent cytotoxicity (CDC) and/or antibody dependent cellular cytoxicity (ADCC), and kill the tumor cell.

In some embodiments, the antibodies or antigen binding fragments as described herein or ADC derived therefrom have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments as described herein or ADC derived therefrom can induce complement complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments as described herein or ADC derived therefrom do not have a functional Fc region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A. P329G mutations according to EU numbering). In some embodiments, the Fc region has FLAA mutations (F234A and L235A according to EU numbering). In some embodiments, the Fc has SI mutations (S239D and 1332E mutations according to EU numbering). In some embodiments, the Fc has N297A mutation according to EU numbering. In some embodiments, the Fc has YTE mutations (M252Y, S254T and T256E according to EU numbering).

Methods of Making anti-WT1/HLA Antibodies

A synthesized and purified WT1/MHC complex can be used as an immunogen to generate antibodies using standard techniques for polyclonal and monoclonal antibody preparation. In some embodiments, MHC-I VH/VL mice (Detailed descriptions of MHC-I VH/VL mice can be found e.g., in PCT/CN2022/081924) can be immunized with WT1 (126-134) peptide (WT1_{RMF}. RMFPNAPYL, SEQ ID NO: 100) presented specifically by human HLA-A2.

Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target. e.g., a WT1/HLA-A2 complex. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-WT1/HLA antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chain. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant region (e.g., IgG1, IgG2, IgG3, and IgG4).

Identity or homology with respect to an original sequence is usually the percentage of amino acid residues present within the candidate sequence that are identical with a sequence present within the human, humanized, or chimeric anti-WT1/HLA antibody or fragment, after aligning the sequences and introducing gaps, if necessary, to achieve the maximum percent sequence identity, and not considering any conservative substitutions as part of the sequence identity.

Additional modifications to the anti-WT1/HLA antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-WT1/HLA antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fe region (Eu numbering of Fe region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Chimeric Antigen Receptors (CAR)

Chimeric antigen receptors (CARs) combine many facets of normal T cell activation into a single protein. They link an extracellular antigen recognition domain to an intracellular signaling domain, which activates the T cell when an antigen is bound. CARs are typically composed of four regions: an antigen binding domain, an extracellular hinge region, a transmembrane domain, and an intracellular T cell signaling domain.

The antigen binding domain is exposed to the outside of the cell, in the ectodomain portion of the receptor. It interacts with potential target molecules and is responsible for targeting the CAR-T cell to any cell expressing a matching molecule. The antigen binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. The linker between the two chains consists of hydrophilic residues with stretches of glycine and serine in it for flexibility as well as stretches of glutamate and lysine for added solubility. In some embodiments, the antigen binding domain specifically binds to a tumor associated antigen, e.g., BCMA, CD19, CD22, CD30. CD33, CD56, CD123 (also known as IL-3R), CEA, EBV-related antigens (e.g., LMP2). EGFR, GD2, GPC3, HER2, HPV-related antigens (e.g., E6), MAGE antigens. Mesothelin, MUC-1, NY-ESO-1, PSCA, PSMA, ROR1, WT1, or Claudin 18.2. In some embodiments, the antigen binding domain specifically binds to a WT1/MHC complex (e.g., a WT1/HLA-A2 complex). In some embodiments, the antigen binding domain does not bind to the MHC molecule.

The hinge, also called a spacer, is a small structural domain that sits between the antigen binding domain and the cell's outer membrane. An ideal hinge enhances the flexibility of the scFv receptor head, reducing the spatial constraints between the CAR and its target antigen. This promotes antigen binding and synapse formation between the CAR-T cells and target cells. Hinge sequences are often based on membrane-proximal regions from immune molecules including e.g., IgG, CD8, and CD28.

The transmembrane domain is a structural component, consisting of a hydrophobic alpha helix that spans the cell membrane. It anchors the CAR to the plasma membrane, bridging the extracellular hinge and antigen binding domains with the intracellular signaling region. This domain is essential for the stability of the receptor as a whole. Generally, the transmembrane domain from the most membrane-proximal component of the endodomain is used, but different transmembrane domains result in different receptor stability. The CD28 transmembrane domain is known to result in a highly expressed, stable receptor.

The intracellular T cell signaling domain lies in the receptor's endodomain, inside the cell. After an antigen is bound to the external antigen binding domain. CAR receptors cluster together and transmit an activation signal. Then the internal cytoplasmic end of the receptor perpetuates signaling inside the T cell. Normal T cell activation relies on the phosphorylation of immunoreceptor tyrosine-based activation motifs (ITAMs) present in the cytoplasmic domain of CD3-zeta. To mimic this process, CD3-zeta's cytoplasmic domain is commonly used as the main CAR endodomain component. T cells also require co-stimulatory molecules in addition to CD3 signaling in order to persist after activation. For this reason, the endodomains of CAR receptors typically also include one or more chimeric domains from co-stimulatory proteins. Signaling domains from a wide variety of co-stimulatory molecules have been successfully tested, including CD28, CD27, CD134 (OX40), and CD137 (4-1BB).

Various CAR molecules and vectors expressing these CAR molecules can be used in the methods described herein. In some embodiments, the CAR molecules specifically binds to a tumor-associated antigen, e.g., a WT1/HLA-A2 complex.

Exemplary structure of antigen receptors, including the hinge, the transmembrane domain, and the intracellular T cell signaling domain, and methods for engineering and introducing such receptors into cells, are described, for example, in Chandran et al., “T cell receptor-based cancer immunotherapy: Emerging efficacy and pathways of resistance.” Immunological reviews 290.1 (2019): 127-147; Cartellieri, Marc, et al., “Chimeric antigen receptor-engineered T cells for immunotherapy of cancer.” BioMed Research International 2010 (2010); and PCT publication No. WO2017173256A1; US2002/131960, US2013/287748, US2013/0149337, U.S. Pat. Nos. 6,451,995, 7,446,190, 8,252,592; each of which is incorporated herein by reference in its entirety.

The disclosure provides chimeric antigen receptors (CARs) or fragments thereof that specifically bind to a WT1/HLA-A2 complex. The CARs or fragments thereof described herein are capable of binding to a WT1/HLA-A2 complex.

The disclosure provides CARs or fragments thereof, comprising (a) an extracellular antigen-binding domain that specifically recognizes a WT1/HLA-A2 complex; (b) a transmembrane domain; and (c) an intracellular signaling region. In some embodiments, the antigen-binding domain includes a heavy chain variable domain (VH) and a light chain variable domain (VL). In some embodiments, the VH and VL of the CAR or fragments thereof described herein are identical to the VH and the VL of any of the antibodies or antigen binding fragments described herein.

In some embodiments, single-chain variable fragments (scFv) of anti-WT1/HLA antibodies (1G7-scFv, 3E6-scFv, P01264-scFv and ESK1-scFv) were used to generate WT1-targeted CARs.

In some embodiments, the CAR has an anti-WT1/HLA antigen-binding domain (e.g., 1G7-scFv, 3E6-scFv, P01264-scFv or ESK1-scFv). In some embodiments, the CAR has the structure shown in FIG. 1D. In some embodiments, the CAR has the below elements linked in tandem: (1) a CD8α signal peptide, (2) an anti-WT1/HLA antigen-binding domain (e.g., 1G7-scFv, 3E6-scFv, P01264-scFv or ESK1-scFv), (3) a CD8α hinge region, (4) a CD8TM transmembrane domain, (5) a 4-1BB intracellular domain, and (6) a CD3ζ intracellular domain.

### Engineered Cells

The present disclosure provides engineered cells (e.g., immune cells, T cells, NK cells, tumor-infiltrating lymphocytes) that express CAR, and/or various proteins as described herein. These engineered cells can be used to treat various disorders or disease as described herein (e.g., WT1-associated cancer).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. In some embodiments, the cells are mouse lymphocytes and engineered (e.g., transduced) to express the CAR, or antigen-binding fragment thereof. In some embodiments, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a tumor-infiltrating lymphocyte (TIL). In some embodiments, the cell is a T cell, B cell or NK cell. In some embodiments, the cells are human peripheral blood mononuclear cells (PBMCs). In some embodiments, the human PBMCs are CD3+ cells. In some embodiments, the human PBMCs are CD8+ cells.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell. In some embodiments, the T cells are central memory T cells. In some embodiments, the T cells are effector memory T cells. In some embodiments, the T cells are naïve T cells.

In some embodiments, the cell is an NK cell. In some embodiments, preparation of the engineered cells includes one or more culture and/or preparation steps. The cells for introduction of the binding molecule, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (IPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

In some embodiments, the engineered cells (e.g. CAR-T cells) are co-cultured with target cells (e.g., antigen presenting cells) for at least or about 1 hour, 2 hours, 3 hours, 4 hours, 5 hours. 6 hours, 12 hours, 18 hours, 1 day, 2 days, 3 days, or longer, such that the engineered cells (e.g., CAR-T cells) can be activated. In some embodiments, the target cells are Jurkat cells.

In some embodiments, IL-12 and modified IL-12 can be expressed by the engineered cells. For example, the fusion protein comprising the modified IL-12 described herein can be expressed on cell surface of engineered cells, e.g., when the fusion protein is a membrane-tethered protein. In some instances, the fusion protein comprising modified IL-12 described herein can be expressed and secreted, e.g., when the fusion protein is a soluble protein. The expression of IL-12 in the engineered cells provides some additional benefits. For example, it can increase production of IFN-γ, which is the most potent mediator of IL-12 actions, from NK and T cells, stimulate of growth and cytotoxicity of activated NK cells, CD8+ and CD4+ T cells, shift differentiation of CD4+Th0 cells toward the Th1 phenotype, increase antibody-dependent cellular cytotoxicity (ADCC) against tumor cells, and induce IgG and suppression of IgE production from B cells, e.g., by at least or about 1 fold, 2 folds, 3 folds, 4 folds, 5 folds, 10 folds, or 20 folds.

In some embodiments, co-culturing with the target cells can increase cytokine (e.g., IFNγ) secretion of the engineered cells by at least or about 1 fold, 2 folds, 5 folds, 10 folds, 20 folds, 30 folds, 40 folds, 50 folds, 60 folds, 70 folds, 80 folds, 90 folds, 100 folds, 200 folds, 500 folds, 1000 folds, 2000 folds, 5000 folds, 10000 folds, or more as compared to the cytokine secretion level of the engineered cell without co-culturing.

In some embodiments, the cells are human PBMCs and engineered (e.g., transduced) to express the CAR, or antigen-binding fragment thereof.

In some embodiments, when the engineered cells are co-cultured with target cells (e.g., WT1 expressing cells), the engineered cells can increase cytokine (e.g., IFNγ) expression or secretion by at least or about 10%, 20%, 30%, 40%, 50%, 60%, 70%, 80%, 90%, 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, or more. In some embodiments, when the engineered cells are co-cultured with target cells (e.g., WT1 expressing cells), the activated T cell population is increased by at least or about 10%, 20%, 30%, 40%, 50%, 60%, 70%, 80%, 90%, 1 fold, 2 folds, 3 folds, 4 folds, 5 folds, 10 folds, 20 folds, 50 folds, 100 folds, or more. In some embodiments, the T cell activation status can be measured by CD69 expression levels.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWINEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Preparing Engineered Cells

The present disclosure provides a method or process for preparing, manufacturing and/or using the engineered cells for treatment of pathological diseases or conditions.

The cells for introduction of the protein described herein, e.g., CAR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate. In some embodiments, the cells are isolated from mouse lymph nodes.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient's blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the nucleic acid construct further includes a sequence encoding an inhibitory protein The method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the cell is transduced for at least or about 1 hour, 2 hours, 3 hours, 4 hours, 5 hours, 6 hours, 12 hours, 18 hours, 1 day, 2 days, 3 days, 4 days, 5 days, 6 days, 1 week, 2 weeks, 3 weeks, or longer. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells can be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

The present disclosure provides a method to create a personalized anti-tumor immunotherapy. Genetically engineered T cells can be produced from a patient's blood cells. These engineered T cells are then reinfused into the patient as a cellular therapy product.

### Methods of Treatment

The antibodies, antigen-binding fragments thereof, or engineered cells of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered cells expressing CAR, to a subject in need thereof (e.g., a subject having, or identified or diagnosed as having, a cancer).

In one aspect, the disclosure features methods that include administering a therapeutically effective amount of antibodies, antigen-binding fragments thereof, or engineered cells disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is leukemia, breast cancer, ovarian cancer, glioblastoma or soft tissue sarcoma.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies. Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody or ADC is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, engineered cell, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, one engineered cell or pharmaceutical composition (e.g., any of the antibodies, antigen-binding antibody fragments, engineered cells or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months. 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine. IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a HER2 agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, anti-ICOS antibody, anti-CD27 antibody, anti-OX40 antibody, anti-4-1BB antibody, anti-CD40 antibody, and/or an anti-GITR antibody.

In one aspect, the disclosure provides a combination therapy. In some embodiments, the anti-WT1/HLA antibody or antigen-binding fragment thereof (e.g., any antibody described herein) can be administered together with an immunomodulatory drug (e.g., lenalidomide).

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for case of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Compositions including the engineered cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population):the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-WT1/HLA Antibodies

The MHC-I VH/VL mice (detailed descriptions of MHC-I VH/VL mice can be found e.g., in PCT/CN2022/081924, which is incorporated herein by reference in the entirety) were immunized with WT1 (126-134) peptide (WT1_{RMF}, RMFPNAPYL, SEQ ID NO: 100) presented specifically by human HLA-A2. The MHC-I VH/VL mice are mice with humanized light chain immunoglobulin locus, humanized heavy chain immunoglobulin locus, and a humanized major histocompatibility complex (MHC) protein complex.

Antigen-positive immune cells were isolated from immunized mice, and fused with mouse myeloma cells to form hybridoma cells. The hybridoma cells secreting antigen-specific monoclonal antibodies were screened, to obtain antigen-specific antibodies. The heavy chain and light chain variable region sequences of antigen-specific antibodies can also be directly obtained by isolating antigen-positive B cells from immunized mice. For example, single-cell technologies (e.g., Beacon® Optofluidic System, Berkeley Lights Inc.) can be used to screen and isolate plasma cells secreting antigen-specific monoclonal antibodies, followed by reverse transcription and PCR sequencing to obtain antibody variable region sequences. The sequences can then be used to express the antibodies. The specificity of the expressed antibody binding to the WT1_{RMF}/HLA-A2 complex can then be verified by FACS (fluorescence activated cell sorting). Exemplary antibodies obtained by this method included: 1E9, 1G7, 3A6, 3E6, 4F10, and 7B9.

In another experiment, the phage display was performed to screen and find monoclonal antibodies that are specific to the WT1_{RMF}/HLA-A2 complex. Exemplary antibodies obtained by this method included P01199, P01218, and P01264.

The heavy and light chain variable regions of 1E9, 1G7, 3A6, 3E6, 4F10, 7B9, P01199, P01218, and P01264 are shown in FIG. 9. FIG. 2 and FIG. 3 show the heavy and light chain CDR sequences of 1E9, 1G7, 3A6, 3E6, 4F10, 7B9, P01199, P01218, and P01264 under Kabat definition and Chothia definition, respectively.

Various IgG1, IgG2 and IgG4 antibodies were made. With respect to the name of the antibodies, when the antibody VH/VL is connected to different isotypes, the isotype is added to the name. For example, if the VH and VL of 1G7 are connected to IgG1 constant regions, the antibody is named as 1G7-IgG1. Examples of other isotypes are as follows: 167-IgG2, 1G7-IgG4. The constant region can also include some mutations. For example, when SI mutations (EU numbering: S239D and 1332E mutations) are introduced into the Fc region of 1G7-IgG1, the resulting antibody is named as 1G7-IgG1-SI.

### Example 2. Binding Affinities of Anti-WT1/HLA Antibodies

The affinity of the anti-WT1/HLA antibodies to WT1_{RMF}/HLA-A2 complex were measured by surface plasmon resonance (SPR) using Biacore (Biacore, INC, Piscataway N.J.) 8K biosensor equipped with pre-immobilized Protein A sensor chips.

Purified anti-WT1/HLA antibodies were diluted to 1 μg/ml and then injected into the Biacore 8K biosensor at 10 μL/min for about 50 seconds to achieve a desired protein density (e.g., about 50 response units (RU)). His-tagged WT1_{RMF}/HLA-A2 complex (WT1 (126-134) peptide presented specifically by human HLA-A2) at concentrations of 200, 100, 50, 25, 6.25 or 1.56 nM were then injected at 30 μL/min for 120 seconds. Dissociation was monitored for 600 seconds. The chip was regenerated after the last injection of each titration with Glycine (pH 2.0, 30 μL/min for 30 seconds).

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore 8K Evaluation Software 3.0. Affinities were deduced from the quotient of the kinetic rate constants (KD=koff/kon).

As a person of ordinary skill in the art would understand, the same method with appropriate adjustments for parameters (e.g., antibody concentration) was performed for each tested antibody. The results for the tested antibodies are summarized in the table below.

TABLE 1
Affinity test results

| Antibody | kon (1/Ms) | koff (1/s) | KD (M) | Results | |
|---|---|---|---|---|---|
| 1E9-IgG1-SI | 7.31E+04 | 1.64E−03 | 2.24E−08 | positive | Binding |
| 1G7-IgG1-SI | 1.59E+05 | 1.51E−03 | 9.49E−09 | positive | Binding |
| 3A6-IgG1-SI | 2.87E+03 | 2.04E−02 | 7.10E−06 | positive | Binding |
| 3E6-IgG1-SI | 1.49E+05 | 4.51E−03 | 3.03E−08 | positive | Binding |
| 4F10-IgG1-SI | 1.54E+06 | 6.64E−04 | 4.30E−10 | positive | Binding |
| 7B9-IgG1-SI | 1.21E+05 | 1.85E−03 | 1.53E−08 | positive | Binding |
| P01199- | 1.47E+05 | 9.87E−03 | 6.74E−08 | positive | Binding |
| IgG1-SI | | | | | |
| P01218- | 5.62E+04 | 9.02E−04 | 1.61E−08 | positive | Binding |
| IgG1-SI | | | | | |
| P01264- | 1.46E+05 | 1.77E−02 | 1.21E−07 | positive | Binding |
| IgG1-SI | | | | | |

The results showed that all nine antibodies showed good binding affinity to WT1_{RMF}/HLA-A2 complex.

### Example 3. Binding Activity Verification of Anti-WT1/HLA Antibodies to T2 Cells Pulsed with WT1_{RMF}

The binding activities of anti-WT1/HLA antibodies to T2 cells (ATCC, Cat #: CRL-1992) pulsed with WT1_{RMF} were verified by flow cytometry.

T2 cells were incubated with WT1_{RMF} (50 μM) for 16 hours at 37° C. in 5% CO₂. After washing, cells were suspended in cold PBS containing serial dilutions of anti-WT1/HLA antibodies (45 nM, 15 nM, 3 nM, 0.6 nM, 0.12 nM, 0.024 nM, 0.0048 nM, 0.00096 nM) for 30 min at 4° C., followed by 30 min incubation with Alexa Fluor® 647 anti-human IgG Fcγ (Jackson Immuno Research Laboratories, Inc., Cat #: 109-606-170).

The cells were collected, and the mean fluorescence intensity (MFI) was determined. A fitting curve was obtained using antibody concentration (nM) as the X-axis and MFI as the Y-axis to obtain the binding EC50 value. The results are shown in the table below.

TABLE 2
Binding of the antibodies to cells

| | EC50(nM) |
|---|---|
| Antibody | Peptide-pulsed T2 cells |
| 1E9-IgGI-SI | 0.7979 |
| 1G7-IgG1-SI | 0.3366 |
| 3A6-IgG1-SI | 2.2560 |
| 3E6-IgG1-SI | 2.0230 |
| 4F10-IgG1-SI | 0.4201 |
| 7B9-IgG1-SI | 0.5535 |
| P01218-IgGI-SI | 3.4840 |
| P01264-IgG1-SI | 0.8960 |
| ESK1 analog | 4.0450 |

ESK1 is a fully human TCR-like monoclonal IgG1 antibody targeting the WT1_{RMF}/HLA-A2 complex. The VH and VL sequences of ESK1 are shown as SEQ ID NO: 111 and SEQ ID NO: 112, respectively.

The results showed that anti-WT1/HLA antibodies 1E9-IgG1-SI, 1G7-IgG1-SI, 3A6-IgG1-SI, 3E6-IgG1-SI, 4F10-IgG1-SI, 7B9-IgG1-SI, P01218-IgG1-SI and P01264-IgG1-SI showed good binding activity to T2 cells pulsed with WT1_{RMF}.

### Example 4. Determination of Binding Sites of Anti-WT1/HLA Antibodies by Alanine Scanning Assay

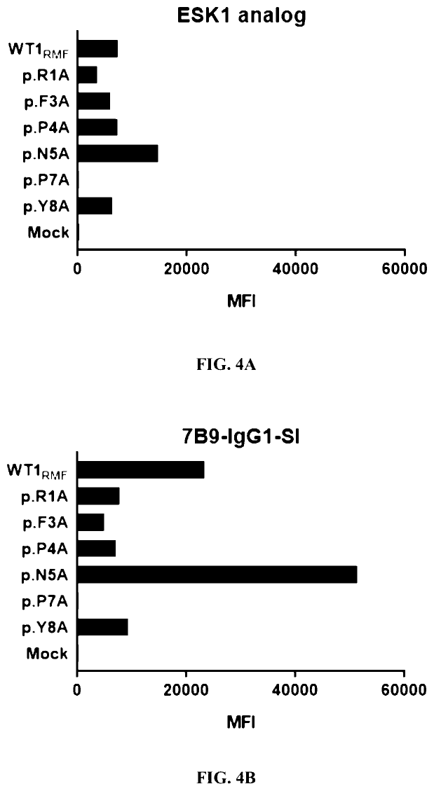

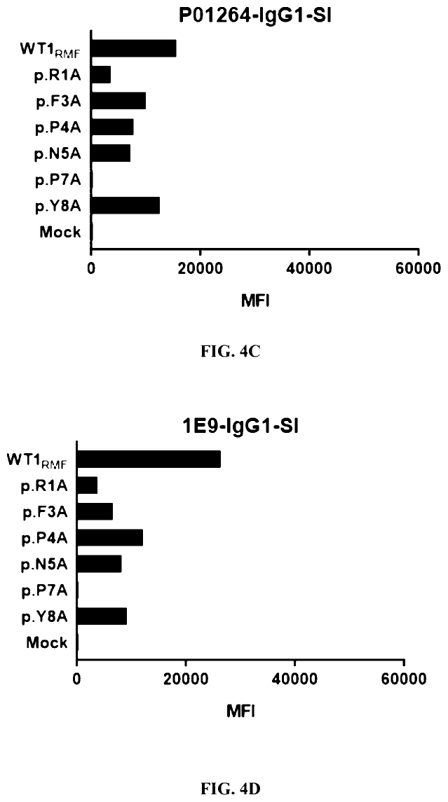

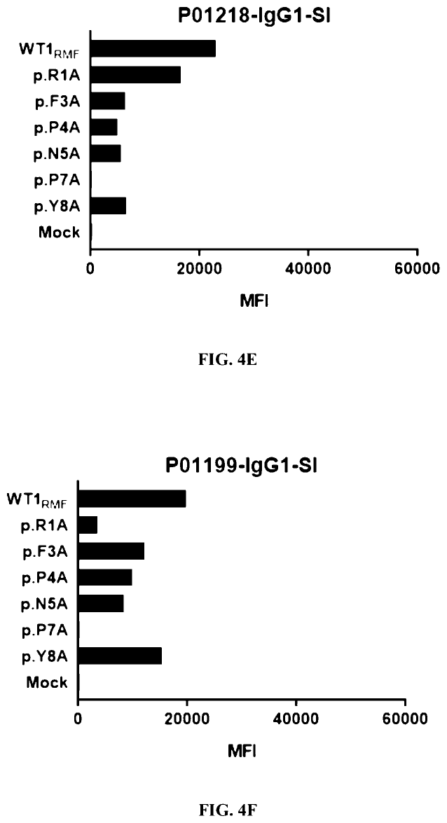

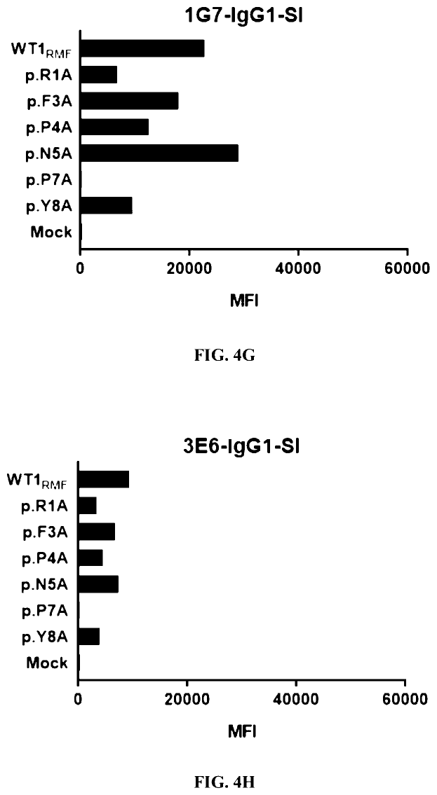

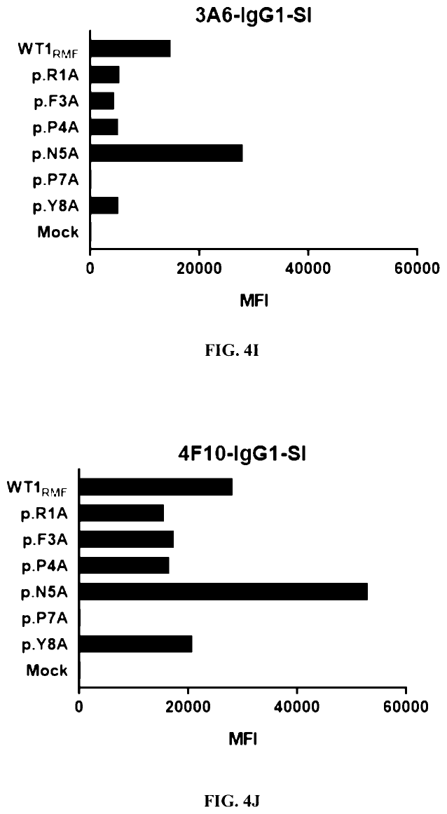

To investigate the binding sites of anti-WT1/HLA antibodies for WT1_{RMF}, an alanine scanning assay was used to determine the recognition epitopes of anti-WT1/HLA antibodies. The amino acids at positions 1, 3, 4, 5, 6, 7 and 8 of WT1_{RMF} were individually replaced with alanine to obtain a series of peptides (as shown in the table below), and flow cytometry was used to measure the binding between T2 cells pulsed with these peptides (peptide concentration: 25 μM) and the anti-WT1/HLA antibodies (antibody concentration: 10 μg/ml). The secondary antibody was Alexa Fluor® 647 anti human IgG Fcγ (Jackson Immuno Research Laboratories, Inc., Cat #: 109-606-170). The mean fluorescence intensity (MFI) results are shown in FIGS. 4A-4J.

TABLE 3
Group of peptides

| Peptides | Sequence | SEQ ID No |
|---|---|---|
| WT1_{RMF} | RMFPNAPYL | 100 |
| p.R1A | AMFPNAPYL | 101 |
| p.F3A | RMAPNAPYL | 102 |
| p.P4A | RMFANAPYL | 103 |
| p.N5A | RMFPAAPYL | 104 |
| p.P7A | RMFPNAAYL | 105 |
| p.Y8A | RMFPNAPAL | 106 |

### Example 5. Binding of Anti-WT1/HLA Antibodies to Potential Off-Target Peptides

Binding of anti-WT1/HLA antibodies to WT1_{RMF} and potential off-target peptides (sequence-similar peptides derived from protein MED13L and PIGQ) was verified by flow cytometry.

Binding of anti-WT1/HLA antibodies to peptides was measured in the TAP1/2 deficient T2 lymphoblastoid cell line by pulsing various peptides onto HLA-A2 cells.

T2 cells were incubated with 25 μM WT1_{RMF}, MED13L peptide (RMFPTPPSL, SEQ ID NO:107) or PIGQ peptide (RMFPGEVAL, SEQ ID NO:108) at 37° C. with 5% CO₂ for 16 hours, respectively. After washing, cells were suspended in cold PBS and incubated with 10 μg/mL anti-WT1/HLA antibodies for 30 min at 4° C., followed by Alexa Fluor® 647 anti-human IgG Fcγ (Jackson Immuno Research Laboratories, Inc., Cat #: 109-606-170) incubate the cells. The results are shown in the table below.

TABLE 4
Binding assay results to potential off-target peptides

| | Percentage of Positive Cells (%) | | | |
|---|---|---|---|---|
| | | WT1_{RMF}-pulsed | MED13L-pulsed | PIGQ-pulsed |
| Antibody | T2 cells | T2 cells | T2 cells | T2 cells |
| ISO | 5.43 | 4.93 | 5.38 | 4.78 |
| ESK1 analog | 5.62 | 88.80 | 96.80 | 78.10 |
| 3A6-IgG1-SI | 5.77 | 70.30 | 5.72 | 5.20 |
| 3E6-IgG1-SI | 6.20 | 30.90 | 5.75 | 5.36 |
| 7B9-IgGI-SI | 4.66 | 93.0 | 7.68 | 5.13 |
| P01199-IgG1-SI | 4.12 | 88.50 | 5.06 | 4.52 |
| P01218-IgGI-SI | 4.51 | 92.80 | 5.06 | 5.42 |
| P01264-IgG1-SI | 4.65 | 75.40 | 5.87 | 4.93 |
| 1E9-IgG1-SI | 4.58 | 94.90 | 5.13 | 4.68 |
| 1G7-IgG1-SI | 5.32 | 92.40 | 5.12 | 5.12 |
| 4F10-IgG1-SI | 7.67 | 95.60 | 13.10 | 7.37 |

PIGQ peptide (SEQ ID NO: 107) and MED13L peptide (SEQ ID NO: 108) are sequence-similar peptides with WT1_{RMF}, and ESK1 analog binds to both PIGQ peptide and MED13L peptide.

The PIGQ peptide occurs ubiquitously in healthy human tissue. Binding to the PIGQ/HLA complex is therefore highly undesirable. Five out of nine amino acids of the PIGQ peptide are identical to the WT1 peptide.

The results showed that anti-WT1/HLA antibodies 3A6-IgG1-SI, 3E6-IgG1-SI, 7B9-IgG1-SI, P01199-IgG1-SI, P01218-IgG1-SI, P01264-IgG1-SI, 1E9-IgG1-SI, 1G7-IgG1-SI and 4F10-IgG1-SI showed good binding affinity to WT1_{RMF}/HLA complex, but not MED13L/HLA complex and PIGQ/HLA complex. In contrary, the positive control ESK1 analog binded not only WT1_{RMF}/HLA complex, but also MED13L-pulsed T2 cells and PIGQ-pulsed T2 cells with high percentage (>50%). Therefore, these 9 antibodies showed specificity for the WT1_{RMF}/HLA complex, but not for the potential off-target peptides PIGQ/HLA complex and MED13L/HLA complex.

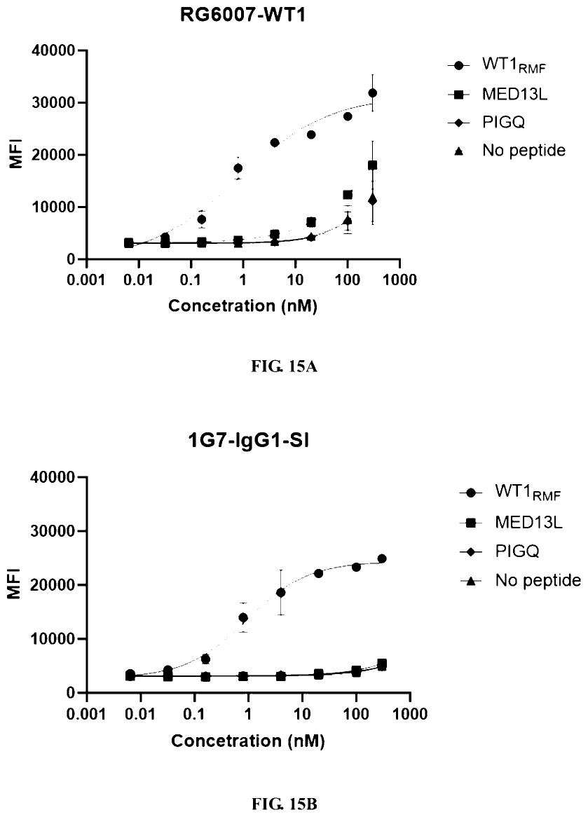

In another similar experiment, the binding activities of 1G7-IgG1-SI or RG6007-WT1 to T2 cells pulsed with 50 μM WT1_{RMF}, MED13L peptide or PIGQ peptide were verified by flow cytometry. The results are shown in FIGS. 15A-15B which indicate that 1G7-IgG1-SI exhibited better binding specificity to WT1_{RMF} than RG6007-WT1.

RG6007 is a T cell bispecific (TCB) trivalent antibody construct targeting HLA-A2-restricted Wilms' tumor gene (WT1) and human CD3e which is in phase I clinical trial at Roche as an intravenous infusion for the treatment of adult patients with hematologic and molecular relapsed/refractory acute myeloid leukemia. The VH (WT1 moiety and CD3 moiety) and VL (WT1 moiety and CD3 moiety) sequences of RG6007 are shown as SEQ ID NO: 117, SEQ ID NO: 118, SEQ ID NO: 119, and SEQ ID NO: 120 respectively. RG6007-WT1 was constructed by linking the VH (WT1 moiety) and VL (WT1 moiety) of RG6007 to IgG1 Fc with SI mutations.

### Example 6. Preparation of Anti-WT1/CD3 Bispecific Antibody

Different bispecific antibodies can be produced with the VH and VL sequences derived from the anti-WT1/HLA antibodies (e.g., 1E9, 1G7, 3A6, 3E6, 4F10, 7B9, P01199, P01218 and P01264) and the anti-CD3 antibodies described herein. Some exemplary antibody structures of the bispecific antibody are shown in FIGS. 1A-1C.

### Structural Form I

WT1-scFv (1E9-scFv, 1G7-scFv, 3A6-scFv, 3E6-scFv, 4F10-scFv, 7B9-scFv. P01199-scFv, P01218-scFv or P01264-scFv) and CD3-scFv (VH SEQ ID NO: 109; VL SEQ ID NO:110) can be paired to form the bispecific antibodies.

The structural form of the bispecific antibody is a BITE (Bispecific T cell Engager) molecule linked to an IgG1 Fc region (with the N297G mutation). The BITE molecule consists of two single-chain variable fragments (scFv) connected in tandem by a flexible fusion linker, where one scFv binds to the T cell surface protein CD3, and another scFv binds to the tumor cell surface antigen WT1. The exemplary structure is shown in FIG. 1B. Exemplary bispecific antibodies obtained included 1E9-CD3, 1G7-CD3, 3A6-CD3, 3E6-CD3, 4F10-CD3, 7B9-CD3, P01199-CD3, P01218-CD3 and P01264-CD3.

### Structural Form II

The bispecific antibody has an anti-WT1 arm comprising a heavy chain and a light chain, and an anti-CD3 arm comprising a variable domain of a heavy chain of heavy-chain antibody (VHH) (e.g. CD3 VHH, SEQ ID NO: 113) connected to the CH2 and CH3 domains of human IgG1. In the Fc region, knobs-into-holes mutations were introduced to the anti-WT1 arm heavy chain and the anti-CD3 arm heavy chain. The exemplary antibody structure is shown in FIG. 1C. Exemplary bispecific antibodies obtained included 1E9-CD3 (VHH), 1G7-CD3 (VHH), 3A6-CD3 (VHH), 3E6-CD3 (VHH), 4F10-CD3 (VHH), 7B9-CD3 (VHH), P01199-CD3 (VHH), P01218-CD3 (VHH) and P01264-CD3 (VHH). For example, 1E9-CD3 (VHH) is a bispecific antibody with IgG1 heavy chain constant region, the heavy chain constant region of CD3 includes knob mutations, and the heavy chain constant region of 1E9 includes hole mutations. The sequences of the light chain constant region, the heavy chain constant region with knob mutations, and the heavy chain constant region with hole mutations are shown in SEQ ID NO: 114, SEQ ID NO: 115 and SEQ ID NO: 116, respectively.

Chinese hamster ovary (CHO) cells were transfected with anti-WT1/HLA×anti-CD3 bispecific antibody expression vectors to express the bispecific antibodies. The CHO cell supernatant containing anti-WT1/HLA×anti-CD3 bispecific antibody molecules was collected and purified to obtain anti-WT1/HLA×anti-CD3 bispecific antibodies. The cell supernatant was collected and purified by Protein A affinity chromatography.

### Example 7. Binding Affinity of Anti-WT1/CD3 Bispecific Antibodies

Binding of anti-WT1/HLA antibodies and anti-WT1/CD3 bispecific antibodies (1E9-CD3, 1G7-CD3, 3A6-CD3, 3E6-CD3, 4F10-CD3, 7B9-CD3, P01199-CD3, P01218-CD3 and P01264-CD3) to HCT116 cells, HCT116-WT1 cells (HCT116 cells expressing the WT1_{RMF}/HLA-A2 complex), Jurkat cells and/or CD3+ T cells were evaluated by flow cytometry. Among these tested cells, the expression of CD3 was not detected in HCT116 and HCT116-WT1 cells.

Specifically, HCT116 cells, HCT116-WT1 cells, Jurkat cells and T cells were incubated with anti-WT1/HLA antibodies or anti-WT1/CD3 bispecific antibodies for 30 min at 4° C., respectively. The cells were then incubated with His-tagged FITC-conjugated antibodies for another 30 min, and the mean fluorescence intensity (MFI) was measured.

The results showed that none of the tested anti-WT1/HLA antibodies and anti-WT1/CD3 bispecific antibodies showed binding to HCT116 cells. By comparison, positive signals were detected on HCT116-WT1 cells, indicating binding. Both CD3+ T cells and Jurkat cells showed binding to nine anti-WT1/CD3 bispecific antibodies, but neither showed binding to anti-WT1/HLA antibodies. Taken together, anti-WT1/CD3 bispecific antibodies can selectively recognize tumor cells (expressing WT1 and HLA-A2) and CD3+ T cells.

### Example 8. Cytotoxicity and T Cell Activation in Engineered WT1 and HLA-A2 Positive Tumor Cells (HCT116-WT1 Cells)

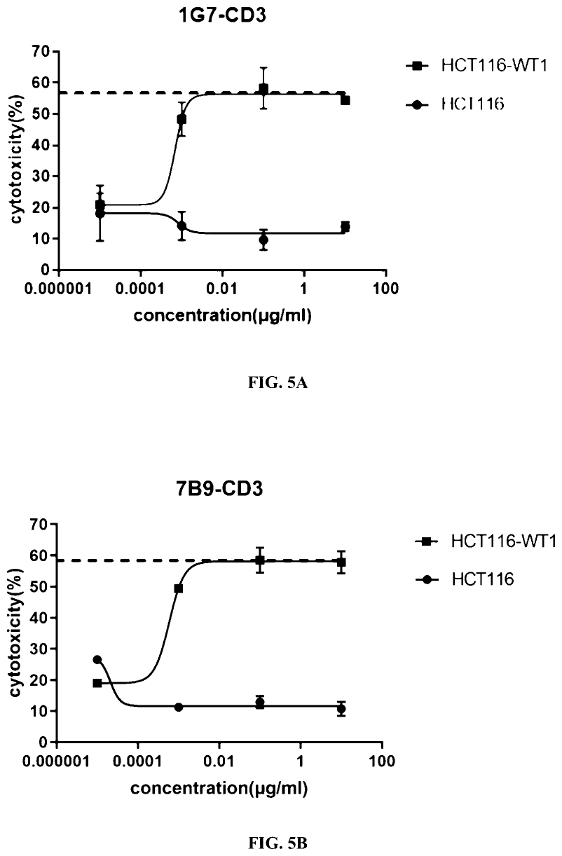

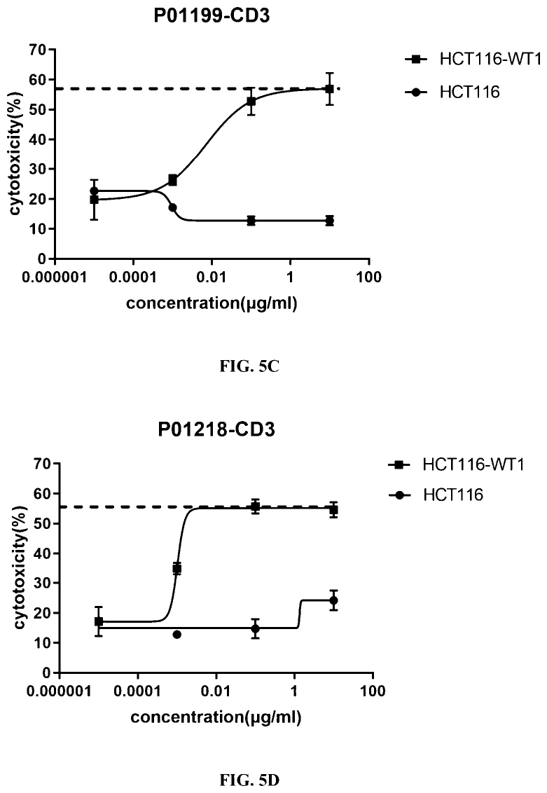

Anti-WT1/CD3 bispecific antibodies (1G7-CD3, 7B9-CD3, P01199-CD3, P01218-CD3, P01264-CD3) at different concentrations (0.00001 μg/mL, 0.001 μg/mL, 0.1 μg/mL, 10 μg/mL) were co-incubated with purified effector cells (CD3+ T cells) and target cells (HCT116-WT1 cells) at a 10:1 ratio (E:T) for 24 hours. In the control group, target cells were replaced by HCT116 cells or T cells to test the specificity of the cell killing. After co-incubating for 24 h, the cells were centrifuged at 500 g for 5 min at 4° C. The supernatant was taken to detect the amount of cytokine IFN-γ. Cell pellets were stained with dead and viable dye for flow cytometry. The results are shown in Table 5 and FIGS. 5A-5D.

TABLE 5
Cytotoxicity of anti-WT1/CD3 bispecific antibodies

| | | Antibody | Cytotoxicity (%) | | | | |
|---|---|---|---|---|---|---|---|
| | | concentration | 1G7- | 7B9- | P01199- | P01218- | P01264- |
| Time | Cells | (μg/mL) | CD3 | CD3 | CD3 | CD3 | CD3 |
| 24 hr | HCT116 | 10⁻⁵ | 18.20 | 26.50 | 22.60 | 17.25 | 25.05 |
| | | 10⁻³ | 14.20 | 11.25 | 17.05 | 12.75 | 16.85 |
| | | 0.1 | 9.69 | 12.85 | 12.70 | 14.70 | 21.45 |
| | | 10 | 13.95 | 10.70 | 12.70 | 24.15 | 18.80 |
| | HCT116-WT1 | 10⁻⁵ | 20.90 | 18.95 | 19.70 | 17.05 | 18.20 |
| | | 10⁻³ | 48.35 | 49.40 | 26.20 | 34.80 | 13.40 |
| | | 0.1 | 58.25 | 58.45 | 52.70 | 55.65 | 55.35 |
| | | 10 | 54.40 | 57.75 | 56.85 | 54.60 | 54.65 |

The results showed that five anti-WT1/CD3 bispecific antibodies induced cytotoxicity against the HCT116-WT1 cells in a dose-dependent manner.

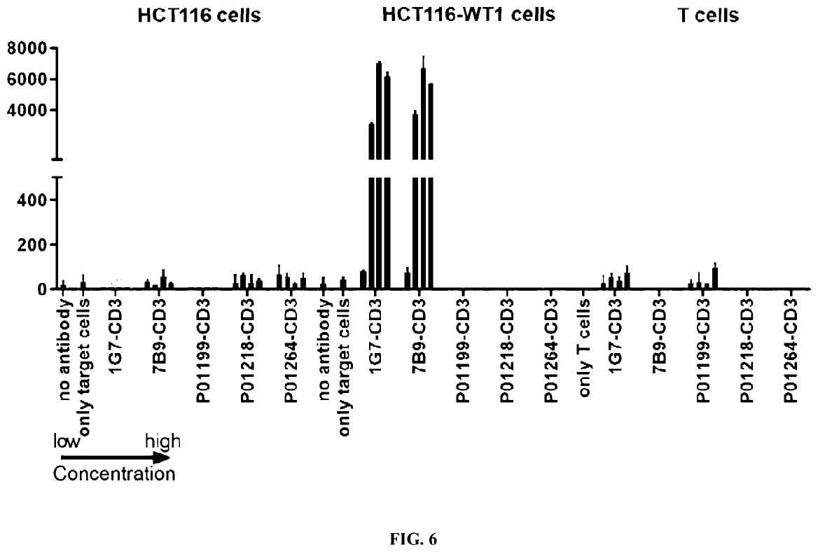

The human IFN-γ cytokine release of the supernatant at 24 h was measured by ELISA, and the results are shown in the table below and FIG. 6.

TABLE 6
IFN-γ cytokine release

| | | INF-γ (pg/mL) | | | | | | | |
|---|---|---|---|---|---|---|---|---|---|
| | Antibody | | Only | Only | | | | | |
| | concentration | No | target | T | 1G7- | 7B9- | P01199- | P01218- | P01264- |
| Cells | (μg/mL) | antibody | cells | cells | CD3 | CD3 | CD3 | CD3 | CD3 |
| HCT116 | 10⁻⁵ | 18.850 | 33.604 | — | 16.925 | 35.165 | 55.405 | 29.090 | 65.285 |
| | 10⁻³ | | | | 19.285 | 18.810 | 31.410 | 62.865 | 55.360 |
| | 0.1 | | | | 45.505 | 55.590 | 30.360 | 29.010 | 27.725 |
| | 10 | | | | 54.830 | 25.220 | 56.715 | 38.845 | 52.560 |
| HCT116- | 10⁻⁵ | 22.820 | 43.956 | — | 82.145 | 76.690 | 36.785 | 62.290 | 19.185 |
| WT1 | 10⁻³ | | | | 3151.350 | 3755.460 | 249.050 | 1086.760 | 32.525 |
| | 0.1 | | | | 7029.310 | 6739.830 | 4865.260 | 6219.520 | 3123.44 |
| | 10 | | | | 6182.650 | 5675.180 | 6942.440 | 4752.630 | 3697.15 |
| T Cells | 10⁻⁵ | — | — | 33.400 | 30.620 | 50.625 | 28.210 | 42.475 | 49.115 |
| | 10⁻³ | | | | 53.475 | 67.200 | 33.925 | 36.150 | 72.460 |
| | 0.1 | | | | 39.720 | 44.900 | 25.820 | 20.460 | 30.125 |
| | 10 | | | | 73.900 | 56.785 | 97.890 | 39.885 | 60.300 |

When HCT116-WT1 cells were used as the target cells, significant induced human IFN-γ secretion could be detected within 24 hours, and a dose-dependent induced IFN-γ release was observed at certain concentrations of the five tested anti-WT1/CD3 bispecific antibodies. By contrast, when HCT116 cells or T cells was used as the target cells, there was no induction of IFN-γ release.

### Example 9. In Vivo Result for Bispecific Antibodies

Anti-WT1/CD3 bispecific antibodies (P01264-CD3 and P01218-CD3) were tested for their effects on tumor growth in vivo in a model of colon carcinoma. About 5×10⁶ HCT116-WT1 cells were injected subcutaneously in B-NDG mice (Biocytogen, Cat #: B-CM-001). On the day of tumor cells injection, each mouse was also injected (by i.v. injection) with PBMC (5E6). When the tumors in the mice reached a volume of 60-120 mm³, the mice were randomly placed into different groups based on tumor volumes and the amount of hCD45. The treatment groups were randomly selected for treatment with anti-WT1/CD3 bispecific antibody (P01264-CD3 or P01218-CD3) by intraperitoneal (i.p.) administration. The control group mice were injected with an equal volume of phosphate buffer saline (PBS). The frequency of administration was twice a week (5 administrations in total). Details are shown in the table below.

TABLE 7
Group assignment

| Group | No. of mice | Bispecific antibody | Dosage | | Route | Total No. of administration |
|---|---|---|---|---|---|---|
| G1 | 6 | PBS | — | | i.p. | 5 |
| G2 | 6 | P01264-CD3 | 0.5 | mg/kg | i.p. | 5 |
| G3 | 6 | P01264-CD3 | 3 | mg/kg | i.p. | 5 |
| G4 | 6 | P01218-CD3 | 0.5 | mg/kg | i.p. | 5 |
| G5 | 6 | P01218-CD3 | 3 | mg/kg | i.p. | 5 |

The lengths of the long axis and the short axis of the tumor were measured and the tumor volumes were calculated as 0.5×(long axis)×(short axis)².

The tumor growth inhibition percentage (TGI %) was calculated using the following formula: TGI (%)=[1−(Ti−T0)/(Vi−V0)]×100%. Ti is the average tumor volume in the treatment group on day i. T0 is the average tumor volume in the treatment group on day zero. Vi is the average tumor volume in the control group on day i. V0 is the average tumor volume in the control group on day zero.

Values are expressed as mean±SEM (standard error of the mean). T-test was performed for statistical analysis. A TGI % higher than 60% indicates clear suppression of tumor growth. P<0.05 is a threshold to indicate significant difference.

The weight of the mice was monitored during the entire treatment period. The weights of mice in different groups all decreased to varying degrees, but the weights of mice in different groups were not obviously different from one another.

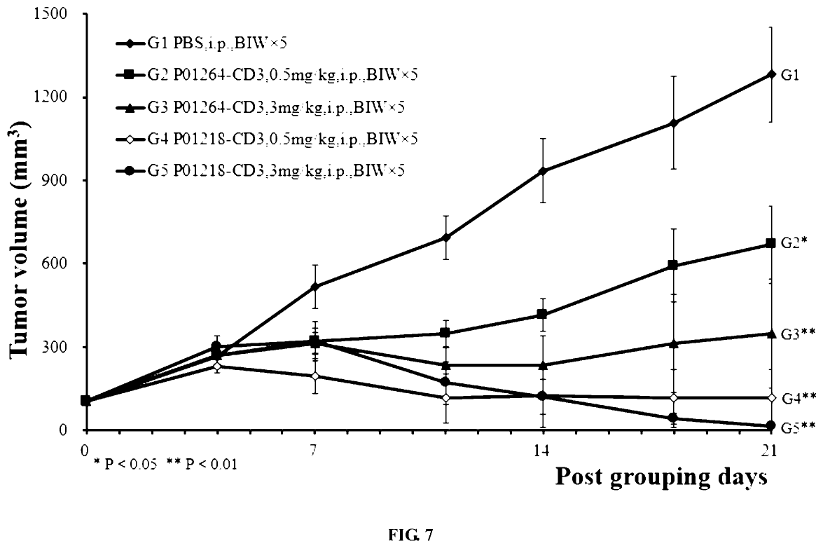

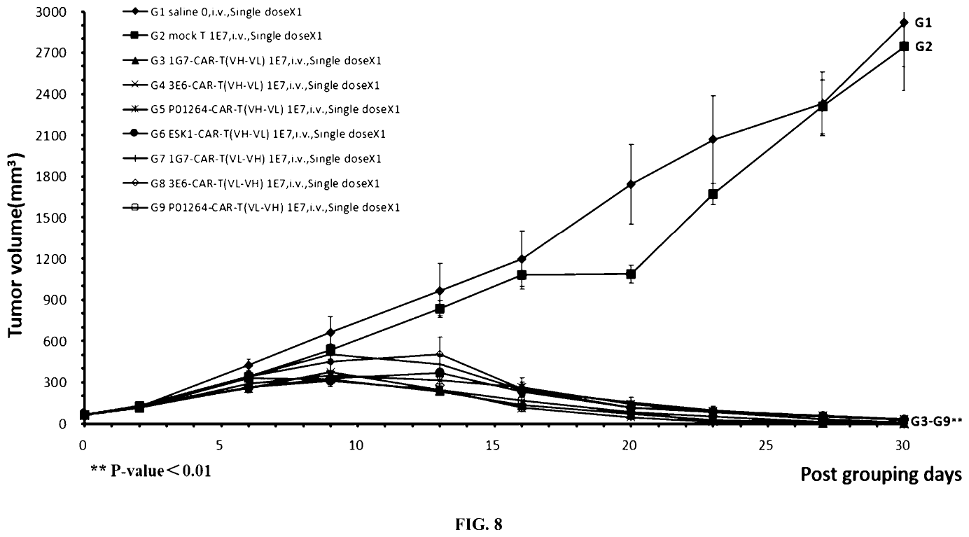

The tumor sizes in groups treated with anti-WT1/CD3 bispecific antibodies are shown in FIG. 7. The table below summarizes the results for this experiment, including the tumor volumes on the day of grouping (day 0), 14 days after grouping (day 14), and at the end of the experiment (day 21); Tumor Growth Inhibition value (TGI); and the statistical differences (P value) of tumor volume and body weight between the treatment and control groups.

TABLE 8
Tumor size changes

| | | | | | | | P value | |
|---|---|---|---|---|---|---|---|---|
| | | Tumor volume(mm³) | | | Tumor- | | Body | Tumor |
| Groups | | Day 0 | Day 14 | Day 21 | free | TGI_{TV} % | weight | Volume |
| Control | G1 | 107 ± 4 | 935 ± 117 | 1282 ± 171 | 0 | N/A | N/A | N/A |
| Treatment | G2 | 107 ± 4 | 415 ± 59 | 669 ± 139 | 0 | 52.1% | 0.055 | 0.020 |
| | G3 | 107 ± 5 | 236 ± 105 | 348 ± 196 | 0 | 79.5% | 0.659 | 0.005 |
| | G4 | 107 ± 6 | 123 ± 112 | 116 ± 104 | 2 | 99.3% | 0.476 | 1.70E−04 |
| | G5 | 107 ± 6 | 121 ± 61 | 14 ± 6 | 2 | 107.9% | 0.305 | 9.00E−04 |

RESULTS: The tumor volumes in treatment groups were smaller than those in the PBS group. The anti-WT1/-CD3 bispecific antibodies P01218-CD3 and P01264-CD3 showed sustained and potent tumor suppression effects. P01218-CD3 even showed better tumor suppression effect at the dosage of 0.5 mg/kg than P01264-CD3 at the dosage of 3 mg/kg.

### Example 10. Anti-Tumor Activity of CAR-T Cells

Single-chain variable fragments (scFv) of anti-WT1/HLA antibodies (1G7-scFv, 3E6-scFv, P01264-scFv and ESK1-scFv) were used to generate WT1-targeted CARs. In some embodiments, the CAR has the structure shown in FIG. 1D. The CARs have the below elements linked in tandem: (1) a CD8α signal peptide, (2) an anti-WT1/HLA antigen-binding moiety (e.g., 1G7-scFv, 3E6-scFv, P01264-scFv or ESK1-scFv), (3) a CD8α hinge region, (4) a CD8TM transmembrane domain, (5) a 4-1BB intracellular domain, and (6) a CD3ζ intracellular domain. The resulting CAR-T cells were named differently based on the order of VH and VL in the scFv. “VH-VL” means that the VH is coupled to CD8α signal peptide and VL is coupled to CD8α hinge region. By contrast, “VL-VH” means that the VL is coupled to CD8α signal peptide and VH is coupled to CD8α hinge region. Thus, the resulting CAR-T cells included: 1G7-CAR-T (VH-VL), 3E6-CAR-T (VH-VL), P01264-CAR-T (VH-VL), ESK1-CAR-T (VH-VL), 1G7-CAR-T (VL-VH), 3E6-CAR-T (VL-VH), P01264-CAR-T (VL-VH), 1E9-CAR-T (VL-VH), 3A6-CAR-T (VL-VH), 4F10-CAR-T (VL-VH), 7B9-CAR-T (VL-VH), P01199-CAR-T (VL-VH), P01218-CAR-T (VL-VH).

### Anti-Tumor Activity of CAR-T Cells in a Colon Cancer Model

CAR-T cells (1G7-CAR-T (VH-VL), 3E6-CAR-T (VH-VL), P01264-CAR-T (VH-VL), ESK1-CAR-T (VH-VL), 1G7-CAR-T (VL-VH), 3E6-CAR-T (VL-VH), P01264-CAR-T (VL-VH)) were tested for their effects on tumor growth in vivo in a model of colon cancer. About 5×10⁶ HCT116-WT1 cells were injected subcutaneously in B-NDG mice. When the tumors in the mice reached a volume of 50-100 mm³, the mice were randomly placed into different groups based on the tumor volume. The mice were then injected with an equal volume of saline, mock T cells or CAR-T cells by intravenous (i.v.) administration the same day after grouping. The frequency of administration was one administration in total. The tumor volume was measured twice a week and the body weights of the mice were measured as well. Animals were ethically sacrificed when the tumor volumes of the mice exceed 3000 mm³. Details are shown in the table below.

TABLE 9
Group assignment

| | No. of | | | | | Total No. of |
|---|---|---|---|---|---|---|
| Group | mice | CAR-T | Dosage | Route | Frequency | administration |
| G1 | 5 | Saline | — | i.v. | Single dose | 1 |
| G2 | 5 | Mock T | 1E7 | i.v. | Single dose | 1 |
| G3 | 5 | 1G7-CAR-T(VH-VL) | 1E7 | i.v. | Single dose | 1 |
| G4 | 5 | 3E6-CAR-T(VH-VL) | 1E7 | i.v. | Single dose | 1 |
| G5 | 5 | P01264-CAR-T(VH-VL) | 1E7 | i.v. | Single dose | 1 |
| G6 | 5 | ESK1-CAR-T(VH-VL) | 1E7 | i.v. | Single dose | 1 |
| G7 | 5 | 1G7-CAR-T(VL-VH) | 1E7 | i.v. | Single dose | 1 |
| G8 | 5 | 3E6-CAR-T(VL-VH) | 1E7 | i.v. | Single dose | 1 |
| G9 | 5 | P01264-CAR-T(VL-VH) | 1E7 | i.v. | Single dose | 1 |

The weights of mice in different groups all increased. On the day of group assignment (Day 0), the average weight of each group was in the range of 18.9 g-19.4 g; At the end of the experiment (Day 30), the average weight of each group was in the range of 19.1 g-22.4 g, the average weight change of each group was in the range of 99.9%-117.6%. The results showed that the tested CARs were well tolerated and were not obviously toxic to the mice.

The tumor size in groups treated with the CAR-T cells are shown in FIG. 8. The table below summarizes the results for this experiment, including the tumor volumes on the day of grouping (day 0), 13 days after the grouping (day 13), 20 days after grouping (day 20), and 30 days after grouping (day 30); the survival rate of the mice; Tumor Growth Inhibition value (TGI); and the statistical differences (P value) of tumor volume and body weight between the treatment and control groups.

TABLE 10
Tumor size changes

| | | | | | | | | | P value | |
|---|---|---|---|---|---|---|---|---|---|---|
| | | Tumor volume (mm³) | | | | Tumor- | | TGI | Body | Tumor |
| Group | | Day 0 | Day 13 | Day 20 | Day 30 | free | Survival | % | weight | Volume |
| Control | G1 | 65 ± 2 | 967 ± 196 | 1745 ± 288 | 2917 ± 493 | 0 | 4/5 | N/A | N/A | N/A |
| Treatment | G2 | 65 ± 2 | 836 ± 54 | 1087 ± 65 | 2745 ± 145 | 0 | 4/5 | 6.0 | 0.007 | 0.749 |
| | G3 | 65 ± 2 | 234 ± 21 | 68 ± 19 | 5 ± 3 | 3 | 5/5 | 102.1 | 0.545 | 2.71E−04 |
| | G4 | 66 ± 3 | 314 ± 25 | 143 ± 23 | 30 ± 9 | 1 | 5/5 | 101.2 | 0.960 | 2.87E−04 |
| | G5 | 66 ± 2 | 244 ± 23 | 48 ± 16 | 0 | 5 | 5/5 | 102.3 | 0.838 | 2.68E−04 |
| | G6 | 65 ± 3 | 370 ± 29 | 117 ± 29 | 33 ± 10 | 1 | 5/5 | 101.1 | 0.910 | 2.88E−04 |
| | G7 | 65 ± 2 | 431 ± 54 | 154 ± 40 | 27 ± 9 | 1 | 5/5 | 101.3 | 0.856 | 2.85E−04 |
| | G8 | 65 ± 2 | 506 ± 123 | 113 ± 30 | 10 ± 10 | 4 | 5/5 | 102.0 | 0.979 | 2.74E−04 |
| | G9 | 66 ± 3 | 248 ± 40 | 82 ± 25 | 0 | 5 | 5/5 | 102.3 | 0.691 | 2.68E−04 |

The tumor volumes in treatment group were smaller than those in the control group (saline and mock T). The CAR-T cells showed sustained and potent tumor suppression effects. Complete tumor suppression was achieved by a serious of CAR-T cell treatments. In addition, the experiment was then continued until 56 days after grouping (Day 56), and all the treatment groups still showed complete tumor suppression.

In another similar experiment, Saline (G1), Mock T cells (G2), CAR T cells 1E9-CAR-T (VL-VH) (G3), 3A6-CAR-T (VL-VH) (G4), 4F10-CAR-T (VL-VH) (G5), P01199-CAR-T (VL-VH) (G6), P01218-CAR-T (VL-VH) (G7) were tested for their effects on tumor growth in vivo in the HCT116-WT1 colon cancer model.

The weight of mice in G3-G8 groups all increased, indicating that the tested CAR-T cells were well tolerated and were not obviously toxic to the mice.

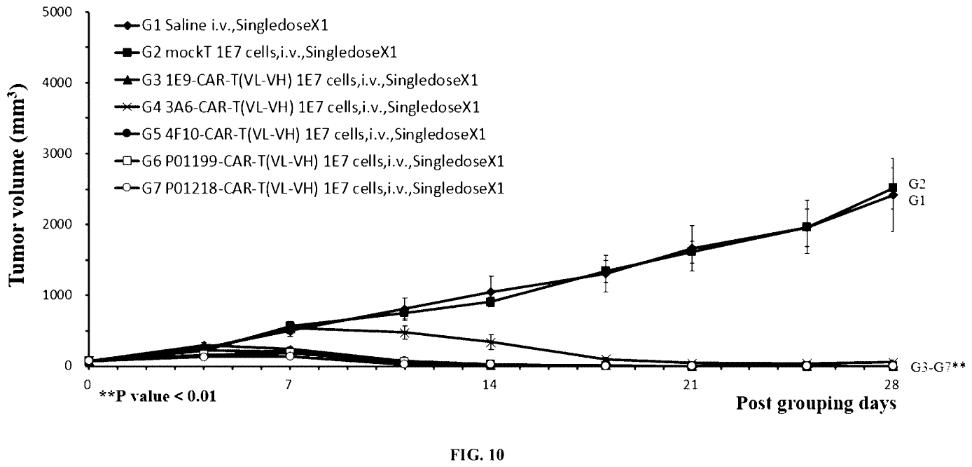

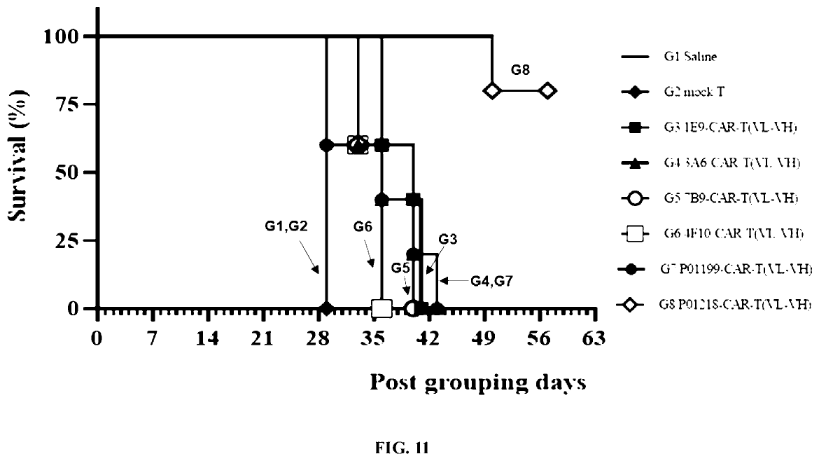

The tumor size in groups treated with the CAR-T cells are shown in FIG. 10. The table below summarizes the results for this experiment, including the tumor volumes on the day of grouping (day 0), 14 days after grouping (day 14), and 28 days after grouping (day 28); the survival and tumor-free of the mice on 56 days after grouping (Day 56); Tumor Growth Inhibition value (TGI); and the statistical differences (P value) of tumor volume between the treatment and control groups.

TABLE 11
Tumor size changes

| | Tumor volume (mm³) | | | Tumor-free | Survival | TGI % | P value |
|---|---|---|---|---|---|---|---|
| Groups | Day 0 | Day 14 | Day 28 | (Day 56) | (Day 56) | (Day 28) | (Day 28) |
| G1 | 73 ± 5 | 1051 ± 215 | 2417 ± 515 | 0 | 0/5 | N/A | N/A |
| G2 | 73 ± 6 | 906 ± 70 | 2515 ± 289 | 0 | 0/5 | −4.2 | 0.872 |
| G3 | 73 ± 6 | 19 ± 5 | 0 | 5 | 5/5 | 103.1 | 0.002 |
| G4 | 73 ± 6 | 340 ± 100 | 55 ± 36 | 2 | 5/5 | 100.8 | 0.002 |
| G5 | 73 ± 6 | 19 ± 6 | 0 | 5 | 5/5 | 103.1 | 0.002 |
| G6 | 73 ± 7 | 27 ± 15 | 0 | 5 | 5/5 | 103.1 | 0.002 |
| G7 | 73 ± 8 | 24 ± 7 | 0 | 5 | 5/5 | 103.1 | 0.002 |

At the end of the experiment, all mice survived and the tumor had regressed in the treatment groups G3-G7, while all mice died in the control groups G1 and G2, indicating that all five anti-WT1 CAR-T cells possess sustained and potent tumor suppression effects.

### Anti-Tumor Effect of the CAR-T Cells in a Model of Leukemia

About 1×10⁶ THP-1-luc cells (Cobioer, Cat #: CBP30129L) were injected in B-NDG mice through the tail vein. In vivo imaging system was used to observe tumor growth in the mice. When the value of the luminescence sign reached 1×10⁶, the mice were randomly placed into different groups. The mice were then injected with an equal volume of Saline, Mock T cells or CAR-T cells by intravenous (i.v.) administration on the day of grouping. Plot of radiance of the mice (p/sec/cm²/sr) was measured twice a week and the body weight of the mice was measured as well. Details are shown in the table below.

TABLE 12
Group assignment

| Group | No. of mice | CAR-T | Dosage | Route | Frequency |
|---|---|---|---|---|---|
| G1 | 5 | Saline | — | i.v. | Single dose |
| G2 | 5 | Mock T | 1E7 | i.v. | Single dose |
| G3 | 5 | 1E9-CAR-T(VL-VH) | 1E7 | i.v. | Single dose |
| G4 | 5 | 3A6-CAR-T(VL-VH) | 1E7 | i.v. | Single dose |
| G5 | 5 | 7B9-CAR-T(VL-VH) | 1E7 | i.v. | Single dose |
| G6 | 5 | 4F10-CAR-T(VL-VH) | 1E7 | i.v. | Single dose |
| G7 | 5 | P01199-CAR-T(VL-VH) | 1E7 | i.v. | Single dose |
| G8 | 5 | P01218-CAR-T(VL-VH) | 1E7 | i.v. | Single dose |

The tumor growth inhibition percentage (TGI %) was calculated using the following formula: TGI (%)=[1−(Ti−T1)/(Ri−R1)]×100%. Ti is the average plot of radiance in the treatment group on Day i. T1 is the average plot of radiance in the treatment group on Day 1. Ri is the plot of radiance in the control group on Day i. R1 is the average plot of radiance in the control group on Day 1.

Values are expressed as mean±SEM (standard error of the mean). T-test was performed for statistical analysis. P<0.05 is a threshold to indicate significant difference.

The weight of the mice was monitored during the entire treatment period. The weights of mice in different groups were not obviously different from one another.

The results showed that all CAR-T cells in treatment groups (G3-G8) showed different tumor suppression effects, wherein P01218-CAR-T (VL-VH) showed the best tumor suppression effect with a TGI of 96.6% on Day 26.

In addition, the experiment was then continued until 57 days after grouping (Day 57), and the survival rate of all groups are shown in FIG. 11. The mice in all six anti-WT1 CAR-T treatment groups exhibited longer survival period than that of the control groups. Especially, P01218-CAR-T (VL-VH) treatment group showed the longest survival period and the highest survival rate among all treatment groups.

### Example 11. Cytotoxic Effect of Anti-WT1/CD3 Bispecific Antibodies

Anti-WT1/CD3 bispecific antibodies (1E9-CD3, 1G7-CD3, 3E6-CD3, 7B9-CD3, P01199-CD3 and P01218-CD3) were serially diluted (5-fold, 9 gradients) with the highest concentration at 100 nM, then co-incubated with purified effector cells (CD3+ T cells) and target cells THP-1 cells (HLA-A2⁺ WT1_{RMF}⁺) or OVCAR3 cells (ATCC, Cat #: HTB-161, HLA-A2⁺ WT1_{RMF}⁺) at a 10:1 ratio (E:T) for 24 or 48 hours to test the cell killing activity, respectively. In the control group, target cells were replaced by HCT116 cells. After co-incubating, the cells were centrifuged at 500 g for 5 min at 4° C. Cell pellets were stained with dead and viable dye for flow cytometry. The supernatant was taken out to detect the expression level of cytokine IFN-γ.

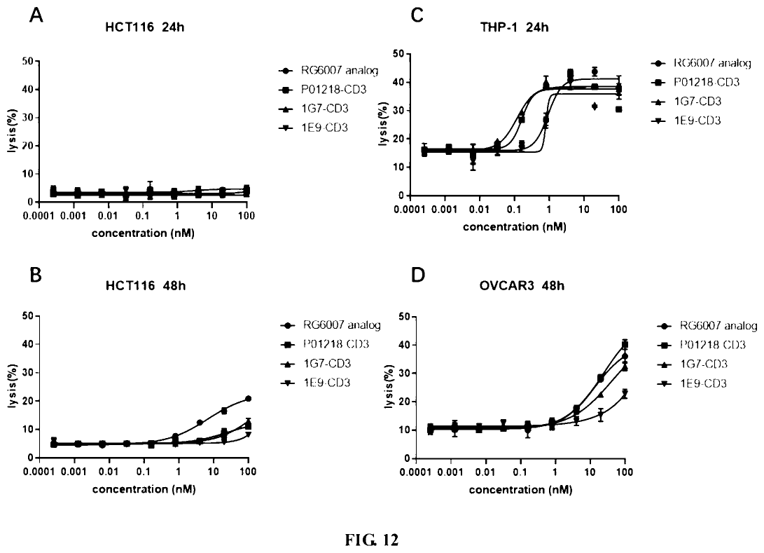

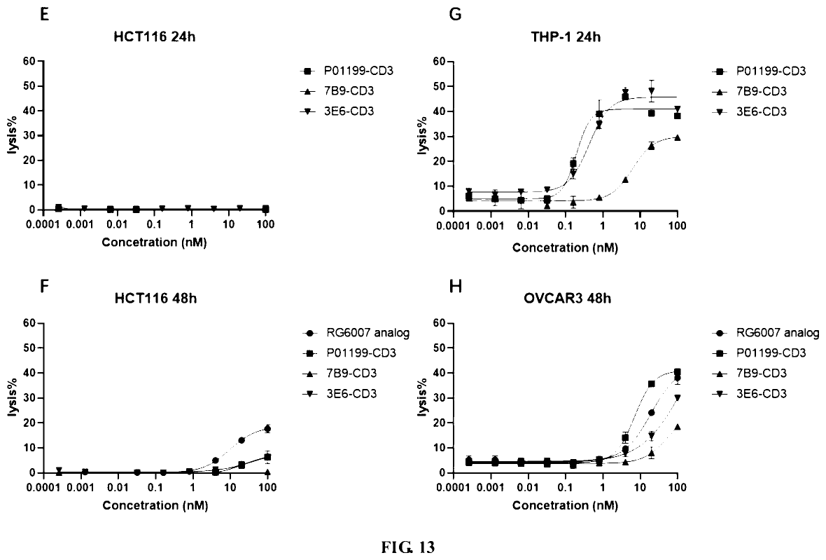

The results are shown in FIG. 12-13, which showed that 1E9-CD3, 1G7-CD3, 3E6-CD3, 7B9-CD3, P01199-CD3 and P01218-CD3 could kill THP-1 cells and OVCAR3 cells, but did not kill negative control HCT116 cells which did not express WT1_{RMF}. However, the positive control RG6007 analog exhibited weak cytotoxicity to HCT116 cells at 48 hours, indicating that 1E9-CD3, 1G7-CD3, 3E6-CD3, 7B9-CD3, P01199-CD3 and P01218-CD3 exhibited better performance of specific targeting and destruction than that of RG6007 analog in OVCAR3 cells.

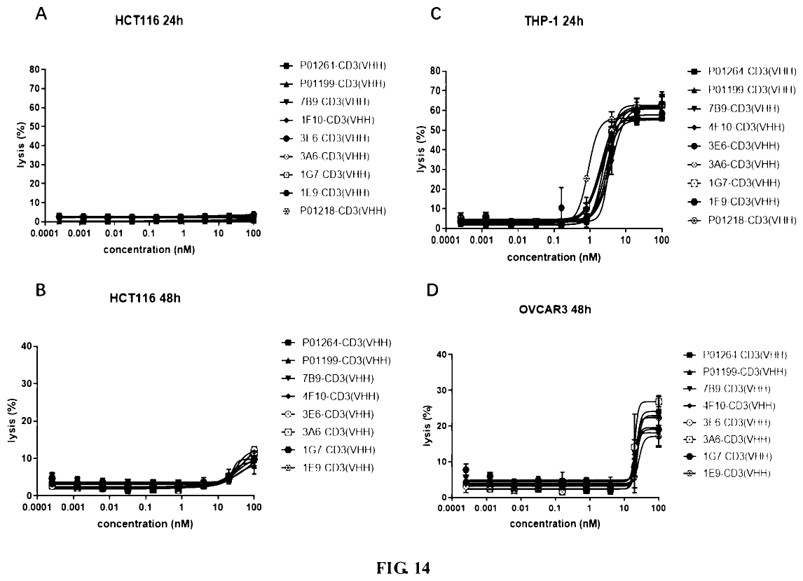

In another similar experiment, the cytotoxicity in nature tumor cells of anti-WT1/CD3 bispecific antibodies 1E9-CD3 (VHH), 1G7-CD3 (VHH), 3A6-CD3 (VHH), 3E6-CD3 (VHH), 4F10-CD3 (VHH), 7B9-CD3 (VHH), P01199-CD3 (VHH), P01218-CD3 (VHH) and P01264-CD3 (VHH) were tested. The results are shown in FIG. 14, which showed that all nine bispecific antibodies could kill THP-1 cells and/or OVCAR3 cells, and did not kill HCT116 cells which did not express WT1_{RMF}.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to a complex comprising a WT1 peptide and a MHC molecule, comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1-3, respectively, and the selected VL CDRs 1, 2, 3 amino acid sequences are set forth in SEQ ID NOs: 55-57, respectively;
(2) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 58-60, respectively;
(3) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 61-63, respectively;
(4) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 64-66, respectively;
(5) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 67-69, respectively;
(6) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 70-72, respectively;
(7) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 73-75, respectively;
(8) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 76-78, respectively;
(9) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 79-81, respectively;
(10) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 55-57, respectively;
(11) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 58-60, respectively;
(12) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 61-63, respectively;
(13) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 64-66, respectively;
(14) the selected VH CDRs 1, 2, 3 amino acid sequences are set forth in SEQ ID NOs: 40-42, respectively, and the selected VL CDRs 1, 2, 3 amino acid sequences are set forth in SEQ ID NOs: 67-69, respectively;
(15) the selected VH CDRs 1, 2, 3 amino acid sequences are set forth in SEQ ID NOs: 43-45, respectively, and the selected VL CDRs 1, 2, 3 amino acid sequences are set forth in SEQ ID NOs: 70-72, respectively;
(16) the selected VH CDRs 1, 2, 3 amino acid sequences are set forth in SEQ ID NOs: 46-48, respectively, and the selected VL CDRs 1, 2, 3 amino acid sequences are set forth in SEQ ID NOs: 73-75, respectively;
(17) the selected VH CDRs 1, 2, 3 amino acid sequences are set forth in SEQ ID NOs: 49-51, respectively, and the selected VL CDRs 1, 2, 3 amino acid sequences are set forth in SEQ ID NOs: 76-78, respectively; and
(18) the selected VH CDRs 1, 2, 3 amino acid sequences are set forth in SEQ ID NOs: 52-54, respectively, and the selected VL CDRs 1, 2, 3 amino acid sequences are set forth in SEQ ID NOs: 79-81, respectively.

2. The antibody or antigen-binding fragment thereof of claim 1, wherein the VH comprises CDRs 1, 2, 3 with the amino acid sequences set forth in SEQ ID NOs: 1-3, respectively, and the VL comprises CDRs 1, 2, 3 with the amino acid sequences set forth in SEQ ID NOs: 55-57, respectively, according to Kabat definition.

3. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 58-60, respectively, according to Kabat definition.

4. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 61-63, respectively, according to Kabat definition.

5. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 64-66, respectively, according to Kabat definition.

6. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 67-69, respectively, according to Kabat definition.

7. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 70-72, respectively, according to Kabat definition.

8. The antibody or antigen-binding fragment thereof of claim 1, wherein the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 73-75, respectively, according to Kabat definition.

9. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 76-78, respectively, according to Kabat definition.

10. The antibody or antigen-binding fragment thereof of claim 1, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 79-81, respectively, according to Kabat definition.

11. The antibody or antigen-binding fragment thereof of claim 1, wherein the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 55-57, respectively, according to Chothia definition.

12. The antibody or antigen-binding fragment thereof of claim 1, wherein the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 58-60, respectively, according to Chothia definition.

13. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 61-63, respectively, according to Chothia definition.

14. The antibody or antigen-binding fragment thereof of claim 1, wherein the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 64-66, respectively, according to Chothia definition.

15. The antibody or antigen-binding fragment thereof of claim 1, wherein the VH comprises CDRs 1, 2, 3 with the amino acid sequences set forth in SEQ ID NOs: 40-42, respectively, and the VL comprises CDRs 1, 2, 3 with the amino acid sequences set forth in SEQ ID NOs: 67-69, respectively, according to Chothia definition.

16. The antibody or antigen-binding fragment thereof of claim 1, wherein the VH comprises CDRs 1, 2, 3 with the amino acid sequences set forth in SEQ ID NOs: 43-45, respectively, and the VL comprises CDRs 1, 2, 3 with the amino acid sequences set forth in SEQ ID NOs: 70-72, respectively, according to Chothia definition.

17. The antibody or antigen-binding fragment thereof of claim 1, wherein the VH comprises CDRs 1, 2, 3 with the amino acid sequences set forth in SEQ ID NOs: 46-48, respectively, and the VL comprises CDRs 1, 2, 3 with the amino acid sequences set forth in SEQ ID NOs: 73-75, respectively, according to Chothia definition.

18. The antibody or antigen-binding fragment thereof of claim 1, wherein the VH comprises CDRs 1, 2, 3 with the amino acid sequences set forth in SEQ ID NOs: 49-51, respectively, and the VL comprises CDRs 1, 2, 3 with the amino acid sequences set forth in SEQ ID NOs: 76-78, respectively, according to Chothia definition.

19. The antibody or antigen-binding fragment thereof of claim 1, wherein the VH comprises CDRs 1, 2, 3 with the amino acid sequences set forth in SEQ ID NOs: 52-54, respectively, and the VL comprises CDRs 1, 2, 3 with the amino acid sequences set forth in SEQ ID NOs: 79-81, respectively, according to Chothia definition.

20. The antibody or antigen-binding fragment thereof of any one of claims 1-19, wherein the antibody or antigen-binding fragment thereof specifically binds to a complex comprising a human WT1 peptide and a MHC molecule.

21. The antibody or antigen-binding fragment thereof of any one of claims 1-20, wherein the WT1 peptide comprises a sequence that is at least 80%, 85%, 90%, 95%, or 100% identical to the amino acid sequence of SEQ ID NO: 100.

22. The antibody or antigen-binding fragment thereof of any one of claims 1-21, wherein the MHC is HLA (e.g., HLA-A2).

23. The antibody or antigen-binding fragment thereof of any one of claims 1-22, wherein the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

24. The antibody or antigen-binding fragment thereof of any one of claims 1-23, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

25. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1-3, respectively, and wherein the VH, when paired with a light chain variable region (VL) comprising the amino acid sequence set forth in SEQ ID NO: 91 binds to a complex comprising a WT1 peptide and a MHC molecule;
(2) an immunoglobulin light chain or a fragment thereof comprising a VL comprising complementarity determining regions (CDRs) 1, 2, and 3 comprising the amino acid sequences set forth in SEQ ID NOs: 55-57, respectively, and wherein the VL, when paired with a VH comprising the amino acid sequence set forth in SEQ ID NO: 82 binds to a complex comprising a WT1 peptide and a MHC molecule;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 92 binds to a complex comprising a WT1 peptide and a MHC molecule;
(4) an immunoglobulin light chain or a fragment thereof comprising a VL comprising complementarity determining regions (CDRs) 1, 2, and 3 comprising the amino acid sequences set forth in SEQ ID NOs: 58-60, respectively, and wherein the VL, when paired with a VH comprising the amino acid sequence set forth in SEQ ID NO: 83 binds to a complex comprising a WT1 peptide and a MHC molecule;
(5) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 93 binds to a complex comprising a WT1 peptide and a MHC molecule;
(6) an immunoglobulin light chain or a fragment thereof comprising a VL comprising complementarity determining regions (CDRs) 1, 2, and 3 comprising the amino acid sequences set forth in SEQ ID NOs: 61-63, respectively, and wherein the VL, when paired with a VH comprising the amino acid sequence set forth in SEQ ID NO: 84 binds to a complex comprising a WT1 peptide and a MHC molecule;
(7) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 94 binds to a complex comprising a WT1 peptide and a MHC molecule;
(8) an immunoglobulin light chain or a fragment thereof comprising a VL comprising CDRs 1, 2, and 3 comprising the amino acid sequences set forth in SEQ ID NOs: 64-66, respectively, and wherein the VL, when paired with a VH comprising the amino acid sequence set forth in SEQ ID NO: 85 binds to a complex comprising a WT1 peptide and a MHC molecule;
(9) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 95 binds to a complex comprising a WT1 peptide and a MHC molecule;
(10) an immunoglobulin light chain or a fragment thereof comprising a VL comprising CDRs 1, 2, and 3 comprising the amino acid sequences set forth in SEQ ID NOs: 67-69, respectively, and wherein the VL, when paired with a VH comprising the amino acid sequence set forth in SEQ ID NO: 86 binds to a complex comprising a WT1 peptide and a MHC molecule;
(11) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 96 binds to a complex comprising a WT1 peptide and a MHC molecule;
(12) an immunoglobulin light chain or a fragment thereof comprising a VL comprising CDRs 1, 2, and 3 comprising the amino acid sequences set forth in SEQ ID NOs: 70-72, respectively, and wherein the VL, when paired with a VH comprising the amino acid sequence set forth in SEQ ID NO: 87 binds to a complex comprising a WT1 peptide and a MHC molecule;
(13) an immunoglobulin heavy chain or a fragment thereof comprising a VH comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 97 binds to a complex comprising a WT1 peptide and a MHC molecule;
(14) an immunoglobulin light chain or a fragment thereof comprising a VL comprising CDRs 1, 2, and 3 comprising the amino acid sequences set forth in SEQ ID NOs: 73-75, respectively, and wherein the VL, when paired with a VH comprising the amino acid sequence set forth in SEQ ID NO: 88 binds to a complex comprising a WT1 peptide and a MHC molecule;
(15) an immunoglobulin heavy chain or a fragment thereof comprising a VH comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 98 binds to a complex comprising a WT1 peptide and a MHC molecule;
(16) an immunoglobulin light chain or a fragment thereof comprising a VL comprising CDRs 1, 2, and 3 comprising the amino acid sequences set forth in SEQ ID NOs: 76-78, respectively, and wherein the VL, when paired with a VH comprising the amino acid sequence set forth in SEQ ID NO: 89 binds to a complex comprising a WT1 peptide and a MHC molecule;
(17) an immunoglobulin heavy chain or a fragment thereof comprising a VH comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 99 binds to a complex comprising a WT1 peptide and a MHC molecule;
(18) an immunoglobulin light chain or a fragment thereof comprising a VL comprising CDRs 1, 2, and 3 comprising the amino acid sequences set forth in SEQ ID NOs: 79-81, respectively, and wherein the VL, when paired with a VH comprising the amino acid sequence set forth in SEQ ID NO: 90 binds to a complex comprising a WT1 peptide and a MHC molecule;
(19) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 91 binds to a complex comprising a WT1 peptide and a MHC molecule;
(20) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 92 binds to a complex comprising a WT1 peptide and a MHC molecule;
(21) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 93 binds to a complex comprising a WT1 peptide and a MHC molecule;
(22) an immunoglobulin heavy chain or a fragment thereof comprising a VH comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 94 binds to a complex comprising a WT1 peptide and a MHC molecule;
(23) an immunoglobulin heavy chain or a fragment thereof comprising a VH comprising complementarity determining regions (CDRs) 1, 2, and 3 comprising the amino acid sequences set forth in SEQ ID NOs: 40-42, respectively, and wherein the VH, when paired with a light chain variable region (VL) comprising the amino acid sequence set forth in SEQ ID NO: 95 binds to a complex comprising a WT1 peptide and a MHC molecule;
(24) an immunoglobulin heavy chain or a fragment thereof comprising a VH comprising complementarity determining regions (CDRs) 1, 2, and 3 comprising the amino acid sequences set forth in SEQ ID NOs: 43-45, respectively, and wherein the VH, when paired with a light chain variable region (VL) comprising the amino acid sequence set forth in SEQ ID NO: 96 binds to a complex comprising a WT1 peptide and a MHC molecule;
(25) an immunoglobulin heavy chain or a fragment thereof comprising a VH comprising complementarity determining regions (CDRs) 1, 2, and 3 comprising the amino acid sequences set forth in SEQ ID NOs: 46-48, respectively, and wherein the VH, when paired with a light chain variable region (VL) comprising the amino acid sequence set forth in SEQ ID NO: 97 binds to a complex comprising a WT1 peptide and a MHC molecule;
(26) an immunoglobulin heavy chain or a fragment thereof comprising a VH comprising complementarity determining regions (CDRs) 1, 2, and 3 comprising the amino acid sequences set forth in SEQ ID NOs: 49-51, respectively, and wherein the VH, when paired with a light chain variable region (VL) comprising the amino acid sequence set forth in SEQ ID NO: 98 binds to a complex comprising a WT1 peptide and a MHC molecule;
(27) an immunoglobulin heavy chain or a fragment thereof comprising a VH comprising complementarity determining regions (CDRs) 1, 2, and 3 comprising the amino acid sequences set forth in SEQ ID NOs: 52-54, respectively, and wherein the VH, when paired with a light chain variable region (VL) comprising the amino acid sequence set forth in SEQ ID NO: 99 binds to a complex comprising a WT1 peptide and a MHC molecule; or

26. The nucleic acid of claim 25, wherein the VH when paired with a VL specifically binds to a complex comprising a WT1 peptide and a MHC molecule, or the VL when paired with a VH specifically binds to a complex comprising a human WT1 peptide and a MHC molecule.

27. The nucleic acid of claim 25 or claim 26, wherein the immunoglobulin heavy chain or the fragment thereof is a human immunoglobulin heavy chain or a fragment thereof (e.g., a human IgG1 heavy chain or a fragment thereof, a human IgG2 heavy chain or a fragment thereof, or a human IgG4 heavy chain or a fragment thereof), and the immunoglobulin light chain or the fragment thereof is a human immunoglobulin light chain or a fragment thereof.

28. The nucleic acid of any one of claims 25-27, wherein the nucleic acid encodes a single-chain variable fragment (scFv), a one-armed antibody, a multi-specific antibody (e.g., a bispecific antibody), or a chimeric antigen receptor (CAR).

29. The nucleic acid of any one of claims 25-28, wherein the nucleic acid is cDNA.

30. A vector comprising one or more of the nucleic acids of any one of claims 25-29.

31. A vector comprising two of the nucleic acids of any one of claims 25-29, wherein the vector encodes the VL region and the VH region that together bind to a complex comprising a WT1 peptide and a MHC molecule.

32. A pair of vectors, wherein each vector comprises one of the nucleic acids of any one of claims 25-29, wherein together the pair of vectors encodes the VL region and the VH region that together bind to a complex comprising a WT1 peptide and a MHC molecule.

33. A cell comprising the vector of claim 30 or 31, or the pair of vectors of claim 32.

34. The cell of claim 33, wherein the cell is a CHO cell.

35. A cell comprising one or more of the nucleic acids of any one of claims 25-29.

36. A cell comprising two of the nucleic acids of any one of claims 25-29.

37. The cell of claim 36, wherein the two nucleic acids together encode the VL region and the VH region that together bind to a complex comprising a WT1 peptide and a MHC molecule.

38. A method of producing an antibody or an antigen-binding fragment thereof, the method comprising
(a) culturing the cell of any one of claims 33-37 under conditions sufficient for the cell to produce the antibody or the antigen-binding fragment; and
(b) collecting the antibody or the antigen-binding fragment produced by the cell.

39. An antibody or antigen-binding fragment thereof that binds to a complex comprising a WT1 peptide and a MHC molecule, comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 82, and the selected VL sequence is SEQ ID NO: 91;
(2) the selected VH sequence is SEQ ID NO: 83, and the selected VL sequence is SEQ ID NO: 92;
(3) the selected VH sequence is SEQ ID NO: 84, and the selected VL sequence is SEQ ID NO: 93;
(4) the selected VH sequence is SEQ ID NO: 85, and the selected VL sequence is SEQ ID NO: 94;
(5) the selected VH sequence is SEQ ID NO: 86, and the selected VL sequence is SEQ ID NO: 95;
(6) the selected VH sequence is SEQ ID NO: 87, and the selected VL sequence is SEQ ID NO: 96;
(7) the selected VH sequence is SEQ ID NO: 88, and the selected VL sequence is SEQ ID NO: 97;
(8) the selected VH sequence is SEQ ID NO: 89, and the selected VL sequence is SEQ ID NO: 98; and
(9) the selected VH sequence is SEQ ID NO: 90, and the selected VL sequence is SEQ ID NO: 99.

40. The antibody or antigen-binding fragment thereof of claim 39, wherein the VH comprises the sequence of SEQ ID NO: 82 and the VL comprises the sequence of SEQ ID NO: 91.

41. The antibody or antigen-binding fragment thereof of claim 39, wherein the VH comprises the sequence of SEQ ID NO: 83 and the VL comprises the sequence of SEQ ID NO: 92.

42. The antibody or antigen-binding fragment thereof of claim 39, wherein the VH comprises the sequence of SEQ ID NO: 84 and the VL comprises the sequence of SEQ ID NO: 93.

43. The antibody or antigen-binding fragment thereof of claim 39, wherein the VH comprises the sequence of SEQ ID NO: 85 and the VL comprises the sequence of SEQ ID NO: 94.

44. The antibody or antigen-binding fragment thereof of claim 39, wherein the VH comprises the sequence of SEQ ID NO: 86 and the VL comprises the sequence of SEQ ID NO: 95.

45. The antibody or antigen-binding fragment thereof of claim 39, wherein the VH comprises the sequence of SEQ ID NO: 87 and the VL comprises the sequence of SEQ ID NO: 96.

46. The antibody or antigen-binding fragment thereof of claim 39, wherein the VH comprises the sequence of SEQ ID NO: 88 and the VL comprises the sequence of SEQ ID NO: 97.

47. The antibody or antigen-binding fragment thereof of claim 39, wherein the VH comprises the sequence of SEQ ID NO: 89 and the VL comprises the sequence of SEQ ID NO: 98.

48. The antibody or antigen-binding fragment thereof of claim 39, wherein the VH comprises the sequence of SEQ ID NO: 90 and the VL comprises the sequence of SEQ ID NO: 99.

49. An antibody or antigen-binding fragment thereof that binds to a complex comprising a WT1 peptide and a MHC molecule, comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 82, and the selected VL sequence is SEQ ID NO: 91;
(2) the selected VH sequence is SEQ ID NO: 83, and the selected VL sequence is SEQ ID NO: 92;
(3) the selected VH sequence is SEQ ID NO: 84, and the selected VL sequence is SEQ ID NO: 93;
(4) the selected VH sequence is SEQ ID NO: 85, and the selected VL sequence is SEQ ID NO: 94;
(5) the selected VH sequence is SEQ ID NO: 86, and the selected VL sequence is SEQ ID NO: 95;
(6) the selected VH sequence is SEQ ID NO: 87, and the selected VL sequence is SEQ ID NO: 96;
(7) the selected VH sequence is SEQ ID NO: 88, and the selected VL sequence is SEQ ID NO: 97;
(8) the selected VH sequence is SEQ ID NO: 89, and the selected VL sequence is SEQ ID NO: 98; and
(9) the selected VH sequence is SEQ ID NO: 90, and the selected VL sequence is SEQ ID NO: 99.

50. The antibody or antigen-binding fragment thereof of any one of claims 39-49, wherein the antibody or antigen-binding fragment thereof specifically binds to a complex comprising a human WT1 peptide and a MHC molecule.

51. The antibody or antigen-binding fragment thereof of any one of claims 39-50, wherein the WT1 peptide comprises a sequence that is at least 80%, 85%, 90%, 95%, or 100% identical to the amino acid sequence of SEQ ID NO: 100.

52. The antibody or antigen-binding fragment thereof of any one of claims 39-51, wherein the MHC is HLA (e.g., HLA-A2).

53. The antibody or antigen-binding fragment thereof of any one of claims 39-52, wherein the antibody or antigen-binding fragment thereof is a human or humanized antibody or antigen-binding fragment thereof, a single-chain variable fragment (scFv), a one-armed antibody and/or a multi-specific antibody (e.g., a bispecific antibody).

54. The antibody or antigen-binding fragment thereof of any one of claims 39-53, wherein the antibody or antigen-binding fragment thereof is a human IgG1 antibody or antigen-binding fragment thereof, a human IgG2 antibody or antigen-binding fragment thereof, or a human IgG4 antibody or antigen-binding fragment thereof.

55. An antibody or antigen-binding fragment thereof that cross-competes with the antibody or antigen-binding fragment thereof of any one of claims 1-24 and 39-54.

56. The antibody or antigen-binding fragment thereof of any one of claims 1-24 and 39-55, wherein the antibody or antigen-binding fragment thereof comprises a fragment crystallizable region (Fc region).

57. A protein construct that binds to a complex comprising a WT1 peptide and a MHC molecule, comprising:
(1) a first functional moiety comprising an antigen-binding fragment thereof of any one of claims 1-24 and 39-55; and
(2) a second functional moiety comprising a T-cell engaging molecule.

58. The protein construct of claim 57, wherein the T-cell engaging molecule (e.g., scFv or VHH) targets human CD3.

59. The protein construct of claim 57 or 58, wherein the WT1 peptide comprises a sequence that is at least 80%, 85%, 90%, 95%, or 100% identical to the amino acid sequence of SEQ ID NO: 100.

60. The protein construct of any one of claims 57-59, wherein the MHC molecule is HLA (e.g., HLA-A2).

61. The protein construct of any one of claims 57-60, wherein the first functional moiety and the second functional moiety are connected via a linker.

62. A protein construct, comprising:
(1) a first functional moiety comprising an antibody or antigen-binding fragment thereof of any one of claims 1-24 and 39-55; and
(2) a second functional moiety comprising a T-cell engaging molecule; and
(3) a third functional moiety comprising a single chain human crystalizable fragment.

63. The protein construct of claim 62, wherein the T-cell engaging molecule is a scFv or VHH targeting human CD3.

64. The protein construct of claim 62 or 63, wherein the first functional moiety, the second functional moiety, and the third functional moiety are connected via one or more linkers.

65. The protein construct of any one of claims 62-64, wherein the WT1 peptide comprises a sequence that is at least 80%, 85%, 90%, 95%, or 100% identical to the amino acid sequence of SEQ ID NO: 100.

66. The protein construct of any one of claims 62-65, wherein the MHC molecule is HLA (e.g., HLA-A2).

67. An antibody-drug conjugate comprising the antibody or antigen-binding fragment thereof of any one of claims 1-24 and 39-55 or the protein construct of any one of claims 57-66 covalently bound to a therapeutic agent.

68. The antibody drug conjugate of claim 67, wherein the therapeutic agent is a cytotoxic or cytostatic agent.

69. An engineered receptor comprising the antigen-binding fragment thereof of any one of claims 1-24 and 39-55.

70. The engineered receptor of claim 69, wherein the engineered receptor further comprises a transmembrane region, and an intracellular signaling domain.

71. The engineered receptor of claim 69 or claim 70, wherein the engineered receptor is a chimeric antigen receptor (“CAR”).

72. The engineered receptor of any one of claims 69-71, wherein the engineered receptor further comprises a hinge region.

73. The engineered receptor of any one of claims 70-72, wherein the transmembrane region comprises a transmembrane region of CD4, CD8, and/or CD28, or a portion thereof.

74. The engineered receptor of any of claims 70-73, wherein the intracellular signaling domain comprises a primary intracellular signaling sequence of an immune effector cell.

75. The engineered receptor of claim 74, wherein the intracellular signaling domain is or comprises a functional signaling domain of CD3 zeta.

76. The engineered receptor of any of claims 70-75, wherein the intracellular signaling domain further comprises a costimulatory signaling domain.

77. The engineered receptor of claim 76, wherein the costimulatory signaling domain comprises a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand.

78. The engineered receptor of claim 76, wherein the costimulatory signaling domain comprises an intracellular signaling domain of 4-1BB and/or CD28.

79. The engineered receptor of any one of claims 69-78, wherein the engineered receptor comprises a signal peptide.

80. The engineered receptor of claim 69, wherein the engineered receptor is a chimeric T cell receptor (“cTCR”).

81. A polynucleotide encoding the engineered receptor of any one of claims 69-80.

82. A vector comprising the polynucleotide of claim 81.

83. The vector of claim 82, wherein the vector is a viral vector.

84. An engineered cell expressing the engineered receptor of any one of claims 69-80.

85. The engineered cell of claim 84, wherein the engineered cell is an immune cell.

86. The engineered cell of claim 85, wherein the immune cell is an NK cell or a T cell.

87. The engineered cell of claim 86, wherein the engineered cell is a T cell.

88. The engineered cell of claim 87, wherein the T cell is selected from the group consisting of cytotoxic T cell, a helper T cell, a natural killer T (NK-T) cell, and a γδT cell.

89. A method for producing an engineered cell, comprising introducing a vector of claim 82 or 83 into a cell in vitro or ex vivo.

90. The method of claim 89, wherein the vector is a viral vector and the introducing is carried out by transduction.

91. A method of treating a subject having cancer, the method comprising administering a therapeutically effective amount of a composition comprising the antibody or antigen-binding fragment thereof of any one of claims 1-24 and 39-56, the protein construct of claims 57-66, the engineered cell of claims 84-88, or the antibody-drug conjugate of claim 67 or 68, to the subject.

92. The method of claim 91, wherein the subject has a solid tumor.

93. The method of claim 91, wherein the cancer is leukemia, breast cancer, ovarian cancer, glioblastoma, colorectal cancer, hematologic malignancy or soft tissue sarcoma.

94. The method of claim 91, wherein the subject is further treated with an effective amount of an anti-4-1BB antibody, an anti-OX40 antibody, an anti-CD40 antibody, anti-PD-1 antibody, anti-CTLA4 antibody, or an anti-PD-L1 antibody.

95. A method of decreasing the rate of tumor growth, the method comprising contacting a tumor cell with an effective amount of a composition comprising an antibody or antigen-binding fragment thereof of any one of claims 1-24 and 39-56, the protein construct of claims 57-66, the engineered cell of claims 84-88, or the antibody-drug conjugate of claim 67 or 68.

96. A method of killing a tumor cell, the method comprising contacting a tumor cell with an effective amount of a composition comprising the antibody or antigen-binding fragment thereof of any one of claims 1-24 and 39-56, the protein construct of claims 57-66, the engineered cell of claims 84-88, or the antibody-drug conjugate of claim 67 or 68.

97. A method of increasing immune response in a subject, the method comprising administering to the subject an effective amount of a composition comprising the antibody or antigen-binding fragment thereof of any one of claims 1-24 and 39-56, the protein construct of claims 57-66, the engineered cell of claims 84-88, or the antibody-drug conjugate of claim 67 or 68.

98. A pharmaceutical composition comprising the antibody or antigen-binding fragment thereof of any one of claims 1-24 and 39-56, the protein construct of claims 57-66, the engineered cell of claims 84-88, or the antibody-drug conjugate of claim 67 or 68, and a pharmaceutically acceptable carrier.

99. The antibody-drug conjugate of claim 67, wherein the drug-to-antibody ratio (DAR) is about 4.

100. An antibody or antigen-binding fragment thereof that binds to a complex comprising a WT1 peptide and a MHC molecule, wherein the antibody or antigen-binding fragment thereof binds specifically to an epitope in the WT1 peptide, wherein the epitope is one or more of the following:
(1) an amino acid residue corresponding to R1 of SEQ ID NO: 100;
(2) an amino acid residue corresponding to F3 of SEQ ID NO: 100;
(3) an amino acid residue corresponding to P4 of SEQ ID NO: 100;
(4) an amino acid residue corresponding to N5 of SEQ ID NO: 100; and
(5) an amino acid residue corresponding to Y8 of SEQ ID NO: 100.
